(19) United States
(12) Patent Application Publication
(10) Pub. No.: US 2022/0134327 A1
(43) Pub. Date: May 5, 2022

(54) BUBBLE-FREE LIQUID FILLING OF FLUIDIC CHAMBERS

(71) Applicant: Lucira Health, Inc., Emeryville, CA (US)

(72) Inventors: Frank B. Myers, III, Richmond, CA (US); Clay D. Reber, Berkeley, CA (US); Taber H. Smith, Saratoga, CA (US); Faisal S. Maniar, San Jose, CA (US)

(21) Appl. No.: 17/436,903

(22) PCT Filed: Mar. 3, 2020
(86) PCT No.: PCT/US2020/020772

§ 371 Date: Sep. 7, 2021

Related U.S. Application Data
(60) Provisional application No. 62/814,143, filed on Mar. 5, 2019.

(51) Int. Cl. B01L 3/00; C12Q 1/6844

(52) U.S. Cl. CPC B01L 3/502; C12Q 1/6846; B01L 2200/026; B01L 2200/0689; B01L 2200/0684; B01L 2300/161; B01L 2200/16; B01L 2300/0654; B01L 2300/0681; B01L 2300/123; B01L 2200/12

(57) ABSTRACT

This invention relates generally to devices, systems, and methods for avoiding bubble formation in a fluidic chamber during filling of the fluidic chamber with a liquid. A first and second piece are operatively coupled to form the fluidic chamber. A protrusion protrudes into a volume of the fluidic chamber such that there is a distance of minimal approach between an apex of the protrusion and a surface of the fluidic chamber. The protrusion forms a channel that extends from one of an inlet and the outlet of the fluidic chamber to the protrusion apex. A maximum distance of travel through the fluidic chamber volume exists between the inlet and the outlet. A cross-sectional area of the fluidic chamber volume increases from the protrusion apex to a transverse plane of the fluidic chamber and decreases from the transverse plane to the other one of the inlet and the outlet.

### INTRODUCTION

Fluidic chambers can contain and facilitate biological and chemical assays that are used to determine one or more characteristics of samples. Oftentimes, to perform such assays within fluidic chambers, assay reactants, including the samples themselves, are transferred into the fluidic chambers via inlets, from an exterior source. Once the assay reactants are located within the fluidic chambers, the assays occur, and products of the assays are produced. These assay products can be analyzed and characterized. Oftentimes, the assay products remain contained within the fluidic chambers during analysis.

### BACKGROUND

As discussed above, many biological and chemical assay systems involve filling fluidic chambers with a liquid and analyzing the products of assays performed using the liquid within the fluidic chambers. In such cases, it is often important to ensure that the liquid fills the fluidic chambers without trapping air bubbles because air bubbles can affect the performance of the assays, reduce effective assay volume, and/or interfere with analysis of the assay products.

In the development of fluidic chambers for biological and chemical assay systems, and particularly for systems including fluidic chambers having nano- or micro-liter volumes, a key challenge is keeping manufacturing costs low while configuring the systems to prevent formation of air bubbles. Using plastic components is a cost-effective manufacturing approach for such assay systems. However, plastics tend to be hydrophobic, which makes it difficult to ensure bubble-free filling. Plastic surfaces can be made more hydrophilic by using plasma treatments, chemical adsorption of hydrophilic molecules, or surface polishing, but these techniques add time and cost to manufacturing.

To combat formation of air bubbles in fluidic chambers, and particularly in low-cost fluidic chambers comprised of plastic components, fluidic chambers can be strategically shaped. However, conventional, low-cost manufacturing techniques restrict ability to shape the geometry of fluidic chambers to prevent formation of air bubbles during filling of the fluidic chambers with a liquid. For example, when using conventional manufacturing techniques such as injection molding to manufacture fluidic chambers, sidewalls of the fluidic chambers are often substantially straight because injection molding of small features generally does not allow for undercuts. As a result, many fluidic chambers comprise five walls that converge with a planar substrate at 90 degree angles. Such 90 degree angles between walls of the fluidic chambers can enable bubble trapping as a liquid fills the fluidic chambers. Therefore, manufacturing limitations prevent fluidic chambers from being configured to avoid bubble formation.

In response to these manufacturing limitations that restrict ability to shape the geometry of fluidic chambers to prevent formation of air bubbles, planar assays systems that operate using traditional lateral flow along a single plane have been developed. These planar assay systems more likely to achieve bubble-free loading of fluidic chambers, but they do not allow for interrogation and analysis of bulk assay volumes. Specifically, only surface reactions can be interrogated in fluidic chambers of planar assay systems because the systems are not configured to provide interrogation access from the sides, and thus analysis is generally performed along an axis that is normal to the plane of the system.

In addition to trapping of air bubbles in fluidic chambers during filling of the fluidic chambers with a liquid, air bubbles can also form after the fluidic chambers are filled, during the assays occurring within the fluidic chambers. For example, air bubbles can form during assays through evolution of gaseous products, release of trapped air in lyophilized reagents, and/or release of dissolved gasses. As mentioned above, presence of air bubbles can interfere with analysis of assay products. In particular, presence of air bubbles can interfere with optical analysis of assay products because of the reflective and refractive properties of air bubbles, and because air bubbles can expand, move, or coalesce during the optical analysis, thereby confounding the analysis.

In the planar assay systems described above, it is difficult to remove air bubbles generated during assays because the air bubbles simply rise to the maximum height of the fluidic chamber, where they remain stagnant along a planar surface. This stagnation of air bubbles at the top of the fluidic chamber along the planar surface is particularly problematic in planar systems because, as mentioned above, interrogation and analysis in planar systems is generally performed along an axis that is normal to the plane of the device. Therefore, this axis of interrogation coincides with air bubbles stagnated along the planar surface of the fluidic chamber, thus interfering with the analysis.

Thus, a key challenge in biological and chemical assay systems is development of low-cost, bulk volume fluidic chambers that are configured to prevent bubble formation during filling of the fluidic chambers with a liquid, as well as to remove bubbles that form within the fluidic chambers during assays executed within the fluidic chambers.

### SUMMARY

The disclosed subject matter relates generally to low-cost devices, systems, and methods for avoiding bubble formation in a fluidic chamber during filling of the fluidic chamber with a liquid. The subject devices include a fluidic chamber that includes an inlet, an outlet, and a protrusion that protrudes into a volume of the fluidic chamber. The subject methods include introducing a liquid into the inlet of the fluidic chamber such that the liquid gradually fills the fluidic chamber such that a radius of curvature of a meniscus of the liquid does not surpass a radius of curvature of one or more interior surfaces of the fluidic chamber, thereby preventing bubble formation within the fluidic chamber. In some embodiments, the devices, systems, and methods disclosed herein also enable removal of bubbles that form within the fluidic chamber. Such subject devices further include at least one sloping surface of the fluidic chamber. Such subject methods further include bubbles rising in the fluidic chamber towards the sloping surface, and then traveling along the sloping surface of the fluidic chamber, away from a center of the fluidic chamber, due to buoyant forces.

In one aspect, the disclosure provides an assembly that is configured to avoid bubble formation in a fluidic chamber of the assembly, during filling of the fluidic chamber with a liquid. To avoid bubble formation, the fluidic chamber of such an assembly has one or more radii of curvature that are each greater than a radius of curvature of a meniscus of the liquid filling the fluidic chamber. This fundamental characteristic of the fluidic chamber is accomplished by strategically configuring the assembly as described here. Specifically, the assembly comprises a first piece and a second piece that are operatively coupled to one another to form the fluidic chamber. The first piece includes a first surface, and similarly the second piece includes a second surface. The first piece also includes a protrusion that is bounded by the first surface of the first piece. The fluidic chamber comprises an inlet, and outlet, and a volume. The volume of the fluidic chamber is bounded by the first surface of the first piece and the second surface of the second piece. The protrusion of the first piece protrudes into the volume of the fluidic chamber such that there is a distance of minimal approach between an apex of the protrusion and the second surface of the second piece of the assembly. The protrusion also forms a channel that extends from one of the inlet and the outlet, to the apex of the protrusion. Due to the protrusion and the channel formed by the protrusion, the inlet and the outlet of the fluidic chamber are positioned in the fluidic chamber such that a maximum distance of travel through the volume of the fluidic chamber exists between the inlet and the outlet. Additionally, a cross-sectional area of the volume of the fluidic chamber increases from the apex of the protrusion to a transverse plane of the fluidic chamber and decreases from the transverse plane of the fluidic chamber to the other one of the inlet and the outlet of the fluidic chamber. This configuration of the fluidic chamber ensures that a radius of curvature of a meniscus of the liquid filling the fluidic chamber has a magnitude that is less than the one or more radii of curvature of the fluidic chamber, thereby preventing formation of bubbles within the fluidic chamber as the fluidic chamber is filled with the liquid.

In addition to this fundamental configuration of the fluidic chamber, the fluidic chamber can comprise additional features that further aid in avoiding formation of bubbles during filling of the fluidic chamber. For instance, in some embodiments, the distance of minimal approach between the apex of the protrusion and the second surface of the second piece is less than a largest dimension of the cross-sectional area of the volume of the fluidic chamber at the transverse plane of the fluidic chamber. This feature further enables prevention of bubble formation in the fluidic chamber because it restricts a size of the radius of curvature of the meniscus of the liquid that fills the fluidic chamber. In further embodiments, the apex of the protrusion can be located diagonally across the volume of the fluidic chamber from the other one of the inlet and the outlet. In even further embodiments, the inlet and the outlet are both formed in the first piece of the assembly. Each of these features maximizes the distance of travel through the volume of the fluidic chamber between the inlet and the outlet, which further aids in bubble prevention during filling of the fluidic chamber with the liquid.

In certain embodiments, the one of the inlet and the outlet from which the channel extends comprises the inlet, and the other of the one of the inlet and the outlet comprises the outlet. In alternative embodiments, the opposite is true. Specifically, in alternative embodiments, the one of the inlet and the outlet from which the channel extends comprises the outlet, and the other of the one of the inlet and the outlet comprises the inlet.

During filling of the fluidic chamber with a liquid, the assembly can have any orientation with respect to gravity, while still preventing formation of bubbles with the fluidic chamber. For instance, during filling of the fluidic chamber with a liquid, in some embodiments the assembly can be oriented such that the second piece is located in the direction of the force of gravity with respect to the first piece. In alternative embodiments, during filling of the fluidic chamber with a liquid, the assembly can be oriented such that the first piece is located in the direction of the force of gravity with respect to the second piece.

Despite the bubble-preventing features of the fluidic chambers described herein, in some embodiments, bubbles may form during filling of a fluidic chamber. Additionally, in certain embodiments, after a fluidic chamber has been filled with a liquid, an assay may be executed within the fluidic chamber causing formation of bubbles within the fluidic chamber. These bubbles may interfere with execution of an assay itself and/or with collection of assay results. Therefore, in addition to configuring a fluidic chamber to avoid bubble formation, in some embodiments it may also be beneficial to configure the fluidic chamber to remove and/or displace bubbles within the fluidic chamber.

In such embodiments, the first surface of the first piece can be configured to slope away from the second surface of the second piece from a sloping point along the first surface towards the other one of the inlet and the outlet of the fluidic chamber. Alternatively, the second surface of the second piece can be configured to slope away from the first surface of the first piece from a second sloping point along the second surface towards the apex of the protrusion of the first piece. As discussed in further detail below, these sloping surfaces enable removal and/or displacement of bubbles within the fluidic chamber, due to buoyancy forces. Therefore, unlike the orientation of the assembly during filling of the fluidic chamber to prevent bubble formation, during removal and/or displacement of bubbles from the fluidic chamber, the assembly should be oriented with respect to gravity such that a sloping surface of the fluidic chamber is located opposite the direction of the force of gravity with respect to the other surface of the fluidic chamber. Specifically, when the first surface of the first piece is configured to slope away from the second surface of the second piece from a sloping point along the first surface towards the other one of the inlet and the outlet of the fluidic chamber, the assembly should be oriented such that the second piece is located in the direction of the force of gravity with respect to the first piece to remove and/or displace bubbles from the fluidic chamber. Conversely, when the second surface of the second piece is configured to slope away from the first surface of the first piece from a second sloping point along the second surface towards the apex of the protrusion of the first piece, the assembly should be oriented such that the first piece is located in the direction of the force of gravity with respect to the second piece to remove and/or displace bubbles from the fluidic chamber.

In another aspect, the disclosure provides another, different embodiment of an assembly that is configured to avoid bubble formation in a fluidic chamber of the assembly, during filling of the fluidic chamber with a liquid. Like the embodiment of the assembly described above, to avoid bubble formation, the fluidic chamber of such an assembly has one or more radii of curvature that are each greater than a radius of curvature of a meniscus of the liquid filling the fluidic chamber. This fundamental characteristic of the fluidic chamber is accomplished slightly differently compared to the embodiment of the assembly described above. The embodiment of the assembly described here also comprises a first piece and a second piece that are operatively coupled to one another to form the fluidic chamber. The first piece includes a first surface, and similarly the second piece includes a second surface. Like the above embodiment of the assembly, the first piece includes a protrusion that is bounded by the first surface of the first piece. However, in the embodiment of the assembly described here, the second piece comprises a second protrusion that is bounded by the second surface of the second piece. The fluidic chamber comprises an inlet, and outlet, and a volume. The volume of the fluidic chamber is bounded by the first surface of the first piece and the second surface of the second piece. The protrusion of the first piece protrudes into the volume of the fluidic chamber such that there is a distance of minimal approach between an apex of the protrusion and the second surface of the second piece of the assembly, and the second protrusion of the second piece protrudes into the volume of the fluidic chamber such that there is a second distance of minimal approach between an apex of the second protrusion and the first surface of the first piece of the assembly. The protrusion forms a channel that extends from one of the inlet and the outlet to the apex of the protrusion, and the second protrusion forms a second channel that extends from the other one of the one of the inlet and the outlet to the apex of the second protrusion. As similarly discussed above, due to the two protrusions and channels, the inlet and the outlet of the fluidic chamber are positioned in the fluidic chamber such that a maximum distance of travel through the volume of the fluidic chamber exists between the inlet and the outlet. Furthermore, a cross-sectional area of the volume of the fluidic chamber increases from the apex of the protrusion to a transverse plane of the fluidic chamber and decreases from the transverse plane of the fluidic chamber to the apex of the second protrusion. This configuration of the fluidic chamber ensures that a radius of curvature of a meniscus of the liquid filling the fluidic chamber has a magnitude that is less than the one or more radii of curvature of the fluidic chamber, thereby preventing formation of bubbles within the fluidic chamber as the fluidic chamber is filled with the liquid.

In addition to this fundamental configuration of the fluidic chamber, as discussed above, the fluidic chamber can further comprise additional features that aid in avoiding formation of bubbles during filling of the fluidic chamber. For instance, in some embodiments, the distance of minimal approach between the apex of the protrusion and the second surface of the second piece and/or the second distance of minimal approach between the apex of the second protrusion and the first surface of the first piece can be less than a largest dimension of the cross-sectional area of the volume of the fluidic chamber at the transverse plane of the fluidic chamber. This feature further enables prevention of bubble formation in the fluidic chamber because it restricts a size of the radius of curvature of the meniscus of the liquid that fills the fluidic chamber. In further embodiments, the apex of the protrusion can be located diagonally across the volume of the fluidic chamber from the apex of the second protrusion. In even further embodiments, the inlet and the outlet are formed in opposite pieces of the assembly. For example, the inlet can be formed in the first piece of the assembly and the outlet can be formed in the second piece of the assembly, or alternatively, the inlet can be formed in the second piece of the assembly and the outlet can be formed in the first piece of the assembly. Each of these features maximizes the distance of travel through the volume of the fluidic chamber between the inlet and the outlet, which further aids in bubble prevention during filling of the fluidic chamber with the liquid.

In certain embodiments, the one of the inlet and the outlet from comprises the inlet, and the other of the one of the inlet and the outlet comprises the outlet. In alternative embodiments, the opposite is true.

As discussed above, during filling of the fluidic chamber with a liquid, the assembly can have any orientation with respect to gravity, while still preventing formation of bubbles with the fluidic chamber. For instance, during filling of the fluidic chamber with a liquid, in some embodiments the assembly can be oriented such that the second piece is located in the direction of the force of gravity with respect to the first piece. In alternative embodiments, during filling of the fluidic chamber with a liquid, the assembly can be oriented such that the first piece is located in the direction of the force of gravity with respect to the second piece.

And as also discussed above, in addition to configuring the fluidic chamber to avoid bubble formation, in some embodiments it may also be beneficial to configure the fluidic chamber to remove and/or displace bubbles within the fluidic chamber. In such embodiments, the first surface of the first piece can be configured to slope away from the second surface of the second piece from a sloping point along the first surface towards the apex of the second protrusion of the second piece. Alternatively, the second surface of the second piece can be configured to slope away from the first surface of the first piece from a second sloping point along the second surface towards the apex of the protrusion of the first piece. These sloping surfaces enable removal and/or displacement of bubbles within the fluidic chamber, due to buoyancy forces. Therefore, unlike the orientation of the assembly during filling of the fluidic chamber to prevent bubble formation, during removal and/or displacement of bubbles from the fluidic chamber, the assembly should be oriented with respect to gravity such that a sloping surface of the fluidic chamber is located opposite the direction of the force of gravity with respect to the other surface of the fluidic chamber. Specifically, when the first surface of the first piece is configured to slope away from the second surface of the second piece from a sloping point along the first surface towards the apex of the second protrusion, the assembly should be oriented such that the second piece is located in the direction of the force of gravity with respect to the first piece to remove and/or displace bubbles from the fluidic chamber. Conversely, when the second surface of the second piece is configured to slope away from the first surface of the first piece from a second sloping point along the second surface towards the apex of the protrusion of the first piece, the assembly should be oriented such that the first piece is located in the direction of the force of gravity with respect to the second piece to remove and/or displace bubbles from the fluidic chamber.

Despite the slight differences in the two embodiments of the fluidic chamber described above, both embodiments have a plurality of features in common. Some of these features further aid in preventing formation of bubbles within the fluidic chamber during filling with a liquid. For instance, in some embodiments, a shape of the volume of the fluidic chamber can substantially comprise a quadrilateral prism. Furthermore, one or more corners of the quadrilateral prism may be radiused. Each of these features help to further ensure that a radius of curvature of a meniscus of the liquid filling the fluidic chamber has a magnitude that is less than the one or more radii of curvature of the fluidic chamber, thereby preventing formation of bubbles within the fluidic chamber as the fluidic chamber is filled with the liquid. In even further embodiments, the first surface of the first piece and the second surface of the second piece have a roughness value of less than 25 micro-inches to prevent formation and catching of bubbles along the surfaces of the fluidic chamber.

There are a variety of ways to form the first and second pieces of the assembly. In some embodiments, at least one of the first piece and the second piece is injection molded. In some embodiments, at least one of the first piece and the second piece is formed by one of replica casting, vacuum-forming, machining, chemical etching, and physical etching. At least one of the first piece and the second piece can comprise one of plastic, metal, and glass. In certain embodiments, at least one of the first piece and the second piece comprises one of a hydrophobic and an oleophobic material such that the contact angle between a liquid filling the fluidic chamber and at least one of the first surface and the second surface of the fluidic chamber is greater than 90 degrees.

There are also a variety of ways to operatively couple the first and second pieces to one another to form the fluidic chamber. In some embodiments, a gasket is located between the first piece and the second piece. In such embodiments, the gasket is operatively coupled to the first piece and the second piece to form fluid seals in the fluidic chamber. In certain embodiments, the gasket can comprise thermoplastic elastomeric (TPE) overmolding. A volume of the gasket can be compressed by 5%-25% when the first piece and the second piece are operatively coupled. In certain embodiments, the first piece and the second piece are operatively coupled by one or more of compression, ultrasonic welding, thermal welding, laser welding, solvent bonding, adhesives, and heat staking.

The fluidic chamber formed by the operative coupling of the first and second pieces of the assembly can take a variety of forms. In certain embodiments, the volume of the fluidic chamber can be between 1 uL and 1000 uL. In a preferred embodiment, the volume of the fluidic chamber can be on the order of 30 uL. In some embodiments, the operative coupling of the first and the second pieces can form a plurality of fluidic chambers. In such embodiments, each of the plurality of fluidic chambers can be in fluidic communication with at least one other fluidic chamber of the plurality of fluidic chambers via at a fluidic connection between one of an inlet and an outlet of the fluidic chamber, and the other of the one of the inlet and the outlet of the at least one other fluidic chamber.

In some embodiments, the one or more fluidic chambers can be used to contain and perform one or more chemical and biological assays. In such embodiments, the fluidic chamber can contain dried or lyophilized reagents. These dried or lyophilized reagents can further comprise reagents such as a nucleic acid amplification enzyme and a DNA primer.

In such embodiment in which the fluidic chamber is used to contain and perform one or more chemical and biological assays, the assembly can further comprise components to interrogate contents of the fluidic chamber. For instance, in some embodiments, the assembly can further comprise a light emitting element configured to interrogate liquid contained in the fluidic chamber. The light emitting element interrogates the liquid contained in the fluidic chamber using light that travels via an interrogation pathway that is orthogonal to the force of gravity. As described in further detail below, this orientation of the interrogation pathway not only enables interrogation of bulk volumes of liquid, but as described in detail below, avoids confounding interference by bubbles within the fluidic chamber, thereby yielding more accurate assay results.

In some embodiments in which the assembly further comprises the light emitting element to interrogate liquid contained in the fluidic chamber, at least a portion of one of the first and second surfaces can comprise a transparent material, and the interrogation pathway via which the light emitting element interrogates the liquid contained in the fluidic chamber can extend through the transparent material. In some further embodiments, the one of the first and second surfaces can be the second surface. The assembly can also further comprise one or more of a light guide, a light filter, and a lens located along the interrogation pathway between the light emitting element and the fluidic chamber.

In yet another aspect, the disclosure provides a method of filling a fluidic chamber of an embodiment of the first assembly (the assembly with a single protrusion) described above, with a liquid. The method includes receiving an embodiment of the first assembly as described above. In particular, the embodiment of the assembly used in the method discussed here is configured such that the one of the inlet and the outlet of the fluidic chamber of the assembly comprises the inlet, and the other one of the inlet and the outlet of the fluidic chamber comprises the outlet. Thus, the embodiment of the assembly used in the method discussed here is configured such that the cross-sectional area of the volume of the fluidic chamber decreases from the transverse plane of the fluidic chamber to the outlet of the fluidic chamber. The method further includes introducing the liquid into the inlet of the fluidic chamber, whereupon the liquid flows from the inlet of the fluidic chamber to the apex of the protrusion of the first piece via the channel formed by the protrusion. Then, upon reaching the apex of the protrusion, the liquid gradually fills the volume of the fluidic chamber such that a radius of curvature of a meniscus of the liquid increases from the apex of the protrusion to the transverse plane of the fluidic chamber, and decreases from the transverse plane of the fluidic chamber to the outlet of the fluidic chamber, but does not surpass a radius of curvature of one or more surfaces of the fluidic chamber, thereby minimizing the trapping of bubbles within the fluidic chamber during filling. In certain embodiments of this method, upon reaching the outlet of the fluidic chamber, the liquid exits the fluidic chamber via the outlet.

In an alternative aspect, the disclosure provides a different method of filling a fluidic chamber of an embodiment of the first assembly (the assembly with a single protrusion) described above, with a liquid. The method includes receiving an embodiment of the first assembly as described above. However, the embodiment of the assembly used in the method discussed here is slightly different than the embodiment of the assembly used in the method discussed above. Specifically, the embodiment of the assembly used in the method discussed here is configured such that the one of the inlet and the outlet of the fluidic chamber of the assembly comprises the outlet, and the other one of the inlet and the outlet of the fluidic chamber comprises the inlet. Thus, the embodiment of the assembly used in the method discussed here is configured such that the cross-sectional area of the volume of the fluidic chamber decreases from the transverse plane of the fluidic chamber to the inlet of the fluidic chamber. The method further includes introducing the liquid into the inlet of the fluidic chamber, whereupon the liquid gradually fills the volume of the fluidic chamber such that a radius of curvature of a meniscus of the liquid increases from the inlet of the fluidic chamber to the transverse plane of the fluidic chamber, and decreases from the transverse plane of the fluidic chamber to the apex of the protrusion, but does not surpass a radius of curvature of one or more surfaces of the fluidic chamber that are normal to the meniscus of the liquid filling the fluidic chamber, thereby minimizing the trapping of bubbles within the fluidic chamber during filling. In certain embodiments of this method, upon reaching the apex of the protrusion, the liquid flows into the channel formed by the protrusion and towards the outlet of the fluidic chamber, and then upon reaching the outlet of the fluidic chamber, the liquid can exit the fluidic chamber via the outlet.

During filling of the fluidic chamber of an embodiment of the first assembly (the assembly with a single protrusion) with a liquid, the assembly can have any orientation with respect to gravity, while still preventing formation of bubbles with the fluidic chamber. For instance, during filling of the fluidic chamber with a liquid, in some embodiments the assembly can be oriented such that the second piece is located in the direction of the force of gravity with respect to the first piece. In alternative embodiments, during filling of the fluidic chamber with a liquid, the assembly can be oriented such that the first piece is located in the direction of the force of gravity with respect to the second piece.

As discussed above, in addition to configuring the fluidic chamber of an embodiment of the first assembly (the assembly with a single protrusion) to avoid bubble formation, in some embodiments it may also be beneficial to configure the fluidic chamber to remove and/or displace bubbles within the fluidic chamber. For instance, the first surface of the first piece of the assembly can slope away from the second surface of the second piece from a sloping point along the first surface towards the outlet of the fluidic chamber. In such embodiments, the method further comprises executing an assay within the fluidic chamber at least in part using the liquid contained within the fluidic chamber, whereupon bubbles formed during execution of the assay rise in the fluidic chamber in the direction opposite the force of gravity, and travel along the sloping first surface of the first piece of the assembly toward the outlet of the fluidic chamber, thereby removing bubbles from the fluidic chamber. During removal of bubbles from the fluidic chamber, the assembly is oriented with respect to gravity such that the second piece is located in the direction of the force of gravity with respect to the first piece.

Alternatively, the second surface of the second piece of the assembly can slope away from the first surface of the first piece from a sloping point along the second surface towards the apex of the protrusion of the first piece. In such embodiments, the method further comprises executing an assay within the fluidic chamber at least in part using the liquid contained within the fluidic chamber, whereupon bubbles formed during execution of the assay rise in the fluidic chamber in the direction opposite the force of gravity, and travel along the sloping second surface of the second piece of the assembly toward the apex of the protrusion of the first piece, thereby displacing bubbles from a center of the volume of the fluidic chamber. During displacement of bubbles from the fluidic chamber, the assembly is oriented with respect to gravity such that the first piece is located in the direction of the force of gravity with respect to the second piece.

In another alternative aspect, the disclosure provides a method of filling a fluidic chamber of an embodiment of the second assembly (the assembly with two protrusions) described above, with a liquid. The method includes receiving an embodiment of the second assembly as described above. In particular, the embodiment of the assembly used in the method discussed here is configured such that the one of the inlet and the outlet of the fluidic chamber of the assembly comprises the inlet, and the other one of the inlet and the outlet of the fluidic chamber comprises the outlet. Thus, the embodiment of the assembly used in the method discussed here is configured such that the cross-sectional area of the volume of the fluidic chamber decreases from the transverse plane of the fluidic chamber to the apex of the second protrusion. The method further includes introducing the liquid into the inlet of the fluidic chamber, whereupon the liquid flows from the inlet of the fluidic chamber to the apex of the protrusion of the first piece via the channel formed by the protrusion. Then, upon reaching the apex of the protrusion, the liquid gradually fills the volume of the fluidic chamber such that a radius of curvature of a meniscus of the liquid increases from the apex of the protrusion to the transverse plane of the fluidic chamber, and decreases from the transverse plane of the fluidic chamber to the apex of the second protrusion of the second piece, but does not surpass a radius of curvature of one or more surfaces of the fluidic chamber that are normal to the meniscus of the liquid filling the fluidic chamber, thereby minimizing the trapping of bubbles within the fluidic chamber during filling. In certain embodiments of this method, upon reaching the apex of the second protrusion, the liquid flows into the second channel formed by the second protrusion and towards the outlet of the fluidic chamber, and then upon reaching the outlet of the fluidic chamber, the liquid can exit the fluidic chamber via the outlet.

In yet another alternative aspect, the disclosure provides a different method of filling a fluidic chamber of an embodiment of the second assembly (the assembly with two protrusions) described above, with a liquid. The method includes receiving an embodiment of the second assembly as described above. However, the embodiment of the second assembly used in the method discussed here is slightly different than the embodiment of the second assembly used in the method discussed above. Specifically, the embodiment of the second assembly used in the method discussed here is configured such that the one of the inlet and the outlet of the fluidic chamber of the assembly comprises the outlet, and the other one of the inlet and the outlet of the fluidic chamber comprises the inlet. Thus, the embodiment of the assembly used in the method discussed here is configured such that the cross-sectional area of the volume of the fluidic chamber decreases from the transverse plane of the fluidic chamber to the apex of the second protrusion. The method further includes introducing the liquid into the inlet of the fluidic chamber, whereupon the liquid flows from the inlet of the fluidic chamber to the apex of the second protrusion of the second piece via the second channel formed by the second protrusion. Then, upon reaching the apex of the second protrusion, the liquid gradually fills the volume of the fluidic chamber such that a radius of curvature of a meniscus of the liquid increases from the apex of the second protrusion to the transverse plane of the fluidic chamber, and decreases from the transverse plane of the fluidic chamber to the apex of the protrusion of the first piece, but does not surpass a radius of curvature of one or more surfaces of the fluidic chamber that are normal to the meniscus of the liquid filling the fluidic chamber, thereby minimizing the trapping of bubbles within the fluidic chamber during filling. In certain embodiments of this method, upon reaching the apex of the protrusion, the liquid flows into the channel formed by the protrusion and towards the outlet of the fluidic chamber, and then upon reaching the outlet of the fluidic chamber, the liquid can exit the fluidic chamber via the outlet.

During filling of the fluidic chamber of an embodiment of the second assembly (the assembly with two protrusions) with a liquid, the assembly can have any orientation with respect to gravity, while still preventing formation of bubbles with the fluidic chamber. For instance, during filling of the fluidic chamber with a liquid, in some embodiments the assembly can be oriented such that the second piece is located in the direction of the force of gravity with respect to the first piece. In alternative embodiments, during filling of the fluidic chamber with a liquid, the assembly can be oriented such that the first piece is located in the direction of the force of gravity with respect to the second piece.

As discussed above, in addition to configuring the fluidic chamber of an embodiment of the second assembly (the assembly with two protrusions) to avoid bubble formation, in some embodiments it may also be beneficial to configure the fluidic chamber to remove and/or displace bubbles within the fluidic chamber. For instance, the second surface of the second piece of the assembly can slope away from the first surface of the first piece from a sloping point along the second surface towards the apex of the protrusion of the first piece. In such embodiments, the method further comprises executing an assay within the fluidic chamber at least in part using the liquid contained within the fluidic chamber, whereupon bubbles formed during execution of the assay rise in the fluidic chamber in the direction opposite the force of gravity, and travel along the sloping second surface of the second piece of the assembly toward the apex of the protrusion of the first piece, thereby displacing bubbles from a center of the volume of the fluidic chamber. During displacement of bubbles from the fluidic chamber, the assembly is oriented with respect to gravity such that the first piece is located in the direction of the force of gravity with respect to the second piece.

Alternatively, the first surface of the first piece of the assembly can slope away from the second surface of the second piece from a sloping point along the first surface towards the apex of the second protrusion of the second piece. In such embodiments, the method further comprises executing an assay within the fluidic chamber at least in part using the liquid contained within the fluidic chamber, whereupon bubbles formed during execution of the assay rise in the fluidic chamber in the direction opposite the force of gravity, and travel along the sloping first surface of the first piece of the assembly toward the apex of the second protrusion of the second piece, thereby displacing bubbles from a center of the volume of the fluidic chamber. During displacement of bubbles from the fluidic chamber, the assembly is oriented with respect to gravity such that the second piece is located in the direction of the force of gravity with respect to the first piece.

In further embodiments of the methods described herein in which the assembly is oriented to remove and/or displace bubbles within the fluidic chamber as discussed above, the assembly can further comprise a light emitting element, and the method can further comprise interrogating the liquid contained in the fluidic chamber using light that travels via an interrogation pathway that is orthogonal to the force of gravity. Due to the orientation of the assembly, bubbles travel along a path of buoyancy in a direction opposite the direction of the force of gravity, and do not interfere with interrogation of the liquid in the fluidic chamber because the path of buoyancy does not coincide with the interrogation pathway that is orthogonal to the force of gravity. This enables more accurate interrogation of the liquid contained in the fluidic chamber. In further embodiments, at least a portion of the second surface of the second piece of the assembly can comprise a transparent material, and interrogating the liquid contained in the fluidic chamber using light that travels via the interrogation pathway that is orthogonal to the force of gravity can comprise the light emitting element emitting light in a direction of the fluidic chamber along the interrogation pathway, through the transparent material and into the fluidic chamber. This transparency of the material further improves the accuracy of the interrogation results.

Various further embodiments apply to any of the methods described herein. For instance, in certain embodiments of the methods described herein, the liquid reaches the outlet of the fluidic chamber when the volume of the fluidic chamber is substantially filled. As used herein, the term “substantially filled” means at least 90% filled. In further embodiments of the methods described herein, the operative coupling of the first and the second pieces of the assembly can form a plurality of fluidic chambers that are in fluidic communication with one another via at least one of the inlet and the outlet of each fluidic chamber, and the liquid can travel between the plurality of fluidic chambers via the at least one of the inlet and the outlet of each fluidic chamber.

### BRIEF DESCRIPTION OF THE DRAWINGS

The present application is further understood when read in conjunction with the appended drawings. For the purpose of illustrating the subject matter, there are shown in the drawings exemplary embodiments of the subject matter; however, the presently disclosed subject matter is not limited to the specific methods, devices, and systems disclosed. In addition, the drawings are not necessarily drawn to scale. In the drawings:

FIG. 1 is a diagram of an assembly for avoiding bubble formation in a fluidic chamber of the assembly, during filling of the fluidic chamber with a liquid, in accordance with an embodiment.

FIG. 2 is a diagram of an assembly for avoiding bubble formation in a fluidic chamber of the assembly, during filling of the fluidic chamber with a liquid, in accordance with an embodiment.

FIG. 3A is a diagram of a first surface of a first piece of an assembly for avoiding bubble formation in a fluidic chamber of the assembly, during filling of the fluidic chamber with a liquid, in accordance with an embodiment.

FIG. 3B is a diagram of a second surface of a second piece of an assembly for avoiding bubble formation in the fluidic chamber of the assembly, during filling of the fluidic chamber with a liquid, in accordance with an embodiment.

FIG. 4A depicts an assembly at a time A during filling of a fluidic chamber of the assembly with a liquid, in accordance with an embodiment.

FIG. 4B depicts an assembly at a time B during filling of a fluidic chamber of the assembly with a liquid, in accordance with an embodiment.

FIG. 4C depicts an assembly at a time C during filling of a fluidic chamber of the assembly with a liquid, in accordance with an embodiment.

FIG. 4D depicts an assembly at a time D during filling of a fluidic chamber of the assembly with a liquid, in accordance with an embodiment.

FIG. 4E depicts an assembly at a time E during filling of a fluidic chamber of the assembly with a liquid, in accordance with an embodiment.

FIG. 4F depicts an assembly at a time F during filling of a fluidic chamber of the assembly with a liquid, in accordance with an embodiment.

FIG. 5A depicts a first fluidic chamber, in accordance with an embodiment.

FIG. 5B depicts a second fluidic chamber, in accordance with an embodiment.

FIG. 5C depicts a third fluidic chamber, in accordance with an embodiment.

FIG. 5D depicts a fourth fluidic chamber, in accordance with an embodiment.

FIG. 5E depicts a fifth fluidic chamber, in accordance with an embodiment.

FIG. 5F depicts a sixth fluidic chamber, in accordance with an embodiment.

FIG. 6A depicts a first fluidic chamber with a sloping surface, in accordance with an embodiment.

FIG. 6B depicts a second fluidic chamber with a sloping surface, in accordance with an embodiment.

FIG. 6C depicts a third fluidic chamber with a sloping surface, in accordance with an embodiment.

FIG. 6D depicts a fourth fluidic chamber with a sloping surface, in accordance with an embodiment.

FIG. 6E depicts a fifth fluidic chamber with a sloping surface, in accordance with an embodiment.

FIG. 6F depicts a sixth fluidic chamber with a sloping surface, in accordance with an embodiment.

FIG. 7A depicts a fluidic chamber configured to avoid bubble formation during filling of the fluidic chamber with a liquid, in accordance with an embodiment.

FIG. 7B depicts the fluidic chamber of FIG. 7A, during filling of the fluidic chamber with a liquid, in accordance with an embodiment.

FIG. 8A depicts a fluidic chamber with a transverse plane, in accordance with an embodiment.

FIG. 8B is a line graph that depicts a relationship between a cross-sectional area A of a volume of a fluidic chamber and a length 1 along the fluidic chamber, in accordance with an embodiment.

FIG. 9 depicts an exemplar fluidic chamber at a plurality of sequential time points during filling of the fluidic chamber with a liquid, in accordance with an embodiment.

FIG. 10 is a cross-section of an assembly for avoiding bubble formation in a fluidic chamber of the assembly, during filling of the fluidic chamber with a liquid, and for interrogation of the liquid contained within the fluidic chamber, in accordance with an embodiment.

### DETAILED DESCRIPTION

Devices, systems, and methods for avoiding bubble formation in a fluidic chamber during filling of the fluidic chamber with a liquid are provided. The subject devices include a fluidic chamber that includes an inlet, an outlet, and a protrusion that protrudes into a volume of the fluidic chamber. The subject methods include introducing a liquid into the inlet of the fluidic chamber such that the liquid gradually fills the fluidic chamber such that a radius of curvature of a meniscus of the liquid does not surpass a radius of curvature of one or more interior surfaces of the fluidic chamber, thereby preventing bubble formation within the fluidic chamber. In some embodiments, the devices, systems, and methods disclosed herein also enable removal of bubbles formed within the fluidic chamber. Such subject devices further include at least one sloping surface of the fluidic chamber. Such subject methods further include bubbles rising in the fluidic chamber towards the sloping surface, and then traveling along the sloping surface of the fluidic chamber, away from a center of the fluidic chamber, due to buoyant forces.

Before the present invention is described in greater detail, it is to be understood that this invention is not limited to particular embodiments described, as such can, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges can independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

It is noted that, as used herein and in the appended claims, the singular forms “a,” “an,” and “the” include plural referents unless the context clearly dictates otherwise. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

Additionally, certain embodiments of the disclosed devices and/or associated methods can be represented by drawings which can be included in this application. Embodiments of the devices and their specific spatial characteristics and/or abilities include those shown or substantially shown in the drawings or which are reasonably inferable from the drawings. Such characteristics include, for example, one or more (e.g., one, two, three, four, five, six, seven, eight, nine, or ten, etc.) of: symmetries about a plane (e.g., a cross-sectional plane) or axis (e.g., an axis of symmetry), edges, peripheries, surfaces, specific orientations (e.g., proximal; distal), and/or numbers (e.g., three surfaces; four surfaces), or any combinations thereof. Such spatial characteristics also include, for example, the lack (e.g., specific absence of) one or more (e.g., one, two, three, four, five, six, seven, eight, nine, or ten, etc.) of: symmetries about a plane (e.g., a cross-sectional plane) or axis (e.g., an axis of symmetry), edges, peripheries, surfaces, specific orientations (e.g., proximal), and/or numbers (e.g., three surfaces), or any combinations thereof.

As will be apparent to those of skill in the art upon reading this disclosure, each of the individual embodiments described and illustrated herein has discrete components and features which can be readily separated from or combined with the features of any of the other several embodiments without departing from the scope or spirit of the present invention. Any recited method can be carried out in the order of events recited or in any other order which is logically possible.

In further describing the subject invention, subject devices for use in practicing the subject devices will be discussed in greater detail, followed by a review of associated methods.

### Devices

Aspects of the subject disclosure include devices for avoiding bubble formation in a fluidic chamber during filling of the fluidic chamber with a liquid. In some embodiments, the devices disclosed herein further include features for removal of bubbles formed within the fluidic chamber.

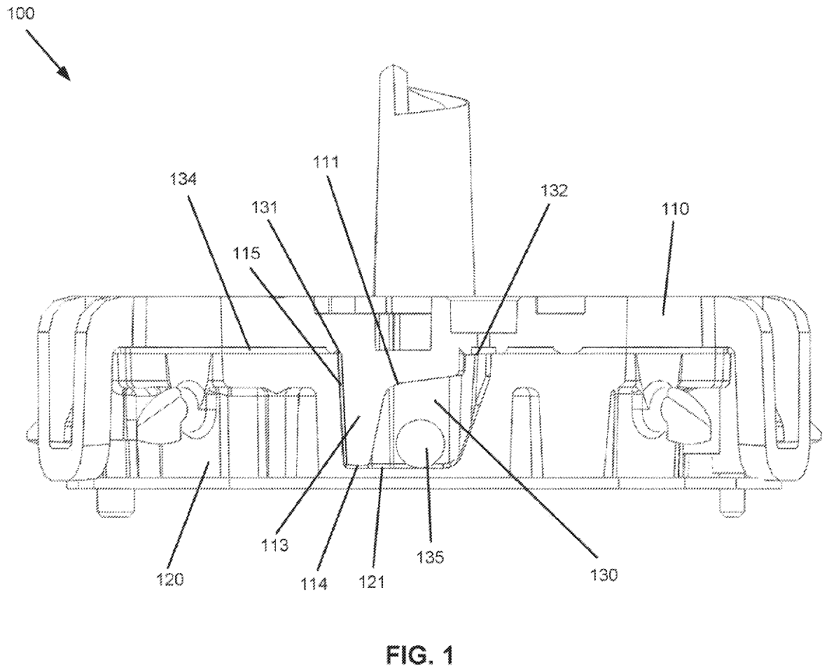

FIG. 1 is a diagram of an assembly 100 for avoiding bubble formation in a fluidic chamber 130 of the assembly 100, during filling of the fluidic chamber 130 with a liquid, in accordance with an embodiment. As shown in FIG. 1, the assembly 100 comprises a minimal number of parts, specifically a first piece 110 and a second piece 120.

In some embodiments, at least one of the first piece 110 and the second piece 120 are injection molded. In alternative embodiments, at least one of the first piece 110 and the second piece 120 may not be injection molded. For example, at least one of the first piece 110 and the second piece 120 can be formed by one of replica casting, vacuum-forming, machining, chemical etching, and/or physical etching. In some embodiments, at least one of the first piece 110 and the second piece 120 may comprise a membrane.

In various embodiments, the assembly 100, including the first piece 110 and the second piece 120, comprises one or more materials including, for example, polymeric materials (e.g., materials having one or more polymers including, for example, plastic and/or rubber), glass, and/or metallic materials. Materials of which any of the assembly 100 can be composed include, but are not limited to: polymeric materials, e.g., elastomeric rubbers, such as natural rubber, silicone rubber, ethylene-vinyl rubber, nitrile rubber, butyl rubber; plastics, such as polytetrafluoroethene or polytetrafluoroethylene (PFTE), including expanded polytetrafluoroethylene (e-PFTE), polyethylene, polyester (Dacron™), nylon, polypropylene, polyethylene, high-density polyethylene (HDPE), polyurethane, polydimethylsiloxane (PDMS); adhesives, such as acrylic adhesive, silicone adhesive, epoxy adhesive, or any combination thereof; metals and metal alloys, e.g., titanium, chromium, aluminum, stainless steel; and/or glass. In various embodiments, the materials are transparent materials and as such, allow light within the visible spectrum to efficiently pass therethrough. In some embodiments, at least one of the first piece 110 and the second piece 120 comprises one of a hydrophobic and/or an oleophobic material, such that a contact angle between a liquid and the material is greater than 90 degrees.

As shown in FIG. 1, the first piece 110 and the second piece 120 of the assembly 100 are configured to be operatively coupled to one another to form the fluidic chamber 130. As used herein, the term “operatively coupled” means connected in a specific way that allows the disclosed devices to operate and/or methods to be carried out effectively in the manner described herein. For example, operatively coupling can include removably coupling or fixedly coupling two or more components. Operatively coupling can also include fluidically, electrically, mateably, and/or adhesively coupling two or more components. As used herein, “removably coupled,” means coupled, e.g., physically, fluidically, and/or electrically coupled, in a manner wherein the two or more coupled components can be un-coupled and then re-coupled repeatedly. The first piece 110 and the second piece 120 can be operatively coupled by one or more of compression, ultrasonic welding, thermal welding, laser welding, solvent bonding, adhesives, and heat staking.

In certain embodiments, the first piece 110 and the second piece 120 are operatively coupled with no components placed between the first piece 110 and the second piece 120. However, in alternative embodiments such as the embodiment depicted in FIG. 1, to operatively couple the first piece 110 and the second piece 120, a gasket 134 can be placed between the first piece 110 and the second piece 120. The gasket 134 can be used to fluidically seal the fluidic chamber 130. In some embodiments, the gasket 134 forms a wall of the fluidic chamber 130. In forming a wall, the gasket 134 can seal and/or extend over an opening at an end of the fluidic chamber 130. As such, the gasket 134 and/or a portion thereof can define an end of the fluidic chamber 130 and/or sealably contain media (e.g., a solid media, a liquid media, a biological sample, an optical property modifying reagent, and/or assay reagents) within the fluidic chamber 130.

For instance, in the embodiment of the assembly 100 depicted in FIG. 1, dried or lyophilized reagents 135 are contained within the fluidic chamber 130. In some embodiments, the dried or lyophilized reagents 135 comprise assay reagents. In further embodiments, the assay reagents comprise a nucleic acid amplification enzyme and a DNA primer. In such embodiments, the assay reagents enable amplification of select nucleic acids present or suspected to be present in a biological sample supplied to the reaction chamber 130. The reagents 135 are dried or lyophilized to prolong shelf stability of the reagents 135 and thus of the assembly 100.

In embodiments in which the gasket 134 is placed between the first piece 110 and the second piece 120 and the first piece 110 and the second piece 120 are operatively coupled, a volume of the gasket 134 can be compressed by 5%-25%. In certain embodiments, the gasket 134 comprises thermoplastic elastomeric (TPE) overmolding. In such embodiments, the gasket 134 can be overmolded on the first piece 110 and/or the second piece 120 to promote sealing of the fluidic chamber 130. In some embodiments, the gasket 134 can be pre-dried to a residual moisture of between 0-0.4% w/w. In a preferred embodiment, the gasket 134 can be pre-dried to a residual moisture of at most 0.2% w/w. Based on this pre-drying of the gasket 134, the assembly 100 can have a shelf stability that exceeds a threshold of 12 months.

In certain embodiments, the gasket 134 can be formed by injection molding. In such embodiments, minimization of flash in the gasket 134 is important because presence of flash in the gasket 134 can disrupt the flow of liquid into the fluidic chamber 130. Specifically, flash in the gasket 134 can disrupt the flow of liquid through the gasket 134 and into the fluidic chamber 130, thereby causing capillary pining effects in the liquid as the liquid enters the fluidic chamber 130. To avoid these undesirable effects, the gasket 134 can be injection molded to a high tolerance.

In alternative embodiments (not shown), the assembly 100 can comprise a single, monolithic piece rather than two separate and operatively coupled pieces such as the first piece 110 and the second piece 120.

As discussed above, the operative coupling of the first piece 110 and the second piece 120 forms the fluidic chamber 130. The first piece 110 of the assembly comprises a first surface 111 and the second piece 120 of the assembly comprises a second surface 121, such that the first surface 111 of the first piece 110 and the second surface 121 of the second piece 120 form the interior surfaces of the fluidic chamber 130. In other words, a volume of the fluidic chamber 130 is bounded by the first surface 111 of the first piece 110 and the second surface 121 of the second piece 120. The fluidic chamber 130 formed by the operative coupling of the first piece 110 and the second piece 120 includes an inlet 131 and an outlet 132.

To prevent bubble formation in the fluidic chamber 130 during filling of the fluidic chamber 130, the first surface 111 of the first piece 110 has one or more primary radii of curvature and the second surface 121 of the second piece 120 has one or more secondary radii of curvature, each of the primary radii of curvature and the secondary radii of curvature being greater than a radius of curvature of a meniscus of a liquid filling the fluidic chamber 130. These radiused surfaces of the fluidic chamber 130 prevent formation and trapping of bubbles in corners of the fluidic chamber 130.

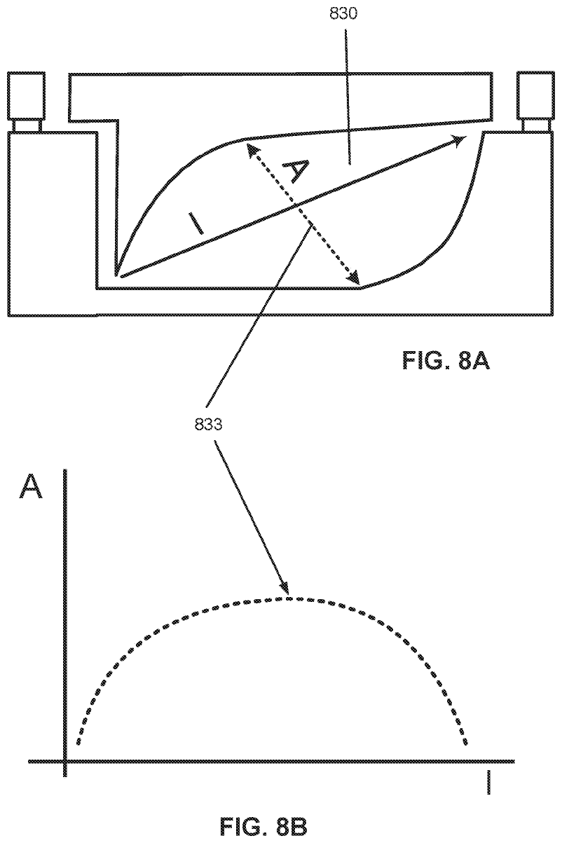

The radiused surfaces of the fluidic chamber 130 that aid in avoiding bubble formation in the fluidic chamber 130 are formed by strategically shaping the fluidic chamber 130 using a protrusion 113. Specifically, as shown in FIG. 1, the first piece 110 of the assembly 100 includes the protrusion 113 bounded by the first surface 111 of the first piece 110. When the first piece 110 and the second piece 120 are operatively coupled to form the fluidic chamber 130, the protrusion 113 protrudes into the fluidic chamber 130 such that there is a distance of minimal approach between an apex of the protrusion 114 and the second surface 121 of the second piece 120. In some embodiments, the distance of minimal approach between an apex of the protrusion 114 and the second surface 121 of the second piece 120 is less than a largest dimension of the cross-sectional area of the volume of the fluidic chamber 130 at a transverse plane of the fluidic chamber 130. The transverse plane of the fluidic chamber 130 is a plane of the fluidic chamber 130 at which a cross-sectional area of the fluidic chamber stops increasing in magnitude and begins decreasing in magnitude. Transverse planes of the fluidic chambers disclosed herein are discussed in further detail below with regard to FIGS. 8A-B.

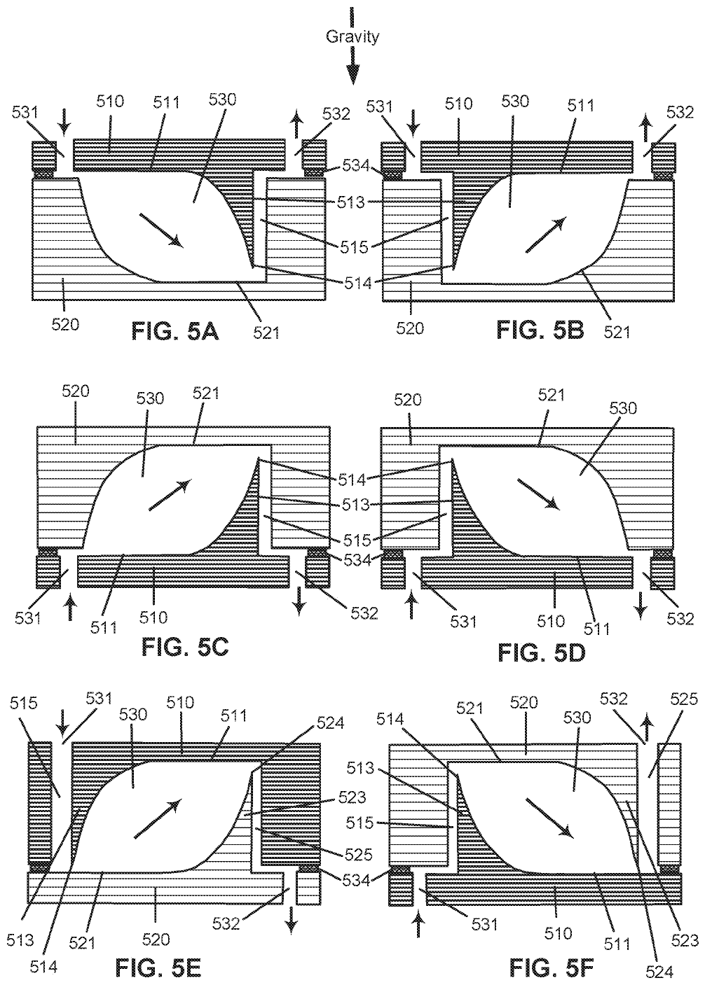

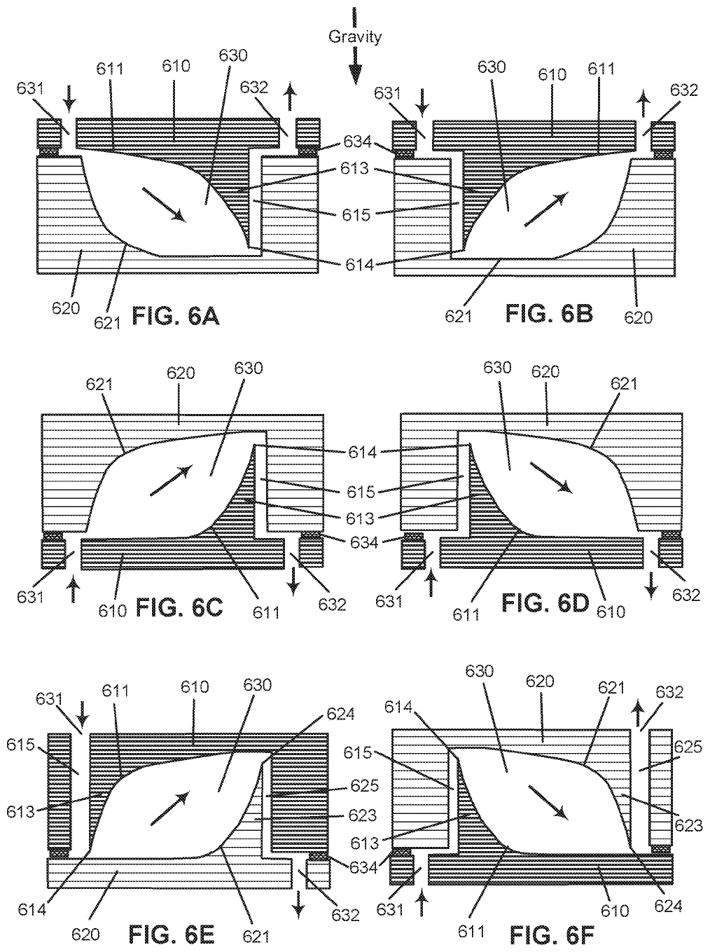

When the first piece 110 and the second piece 120 are operatively coupled to form the fluidic chamber 130, and the protrusion 113 protrudes into the fluidic chamber 130, the protrusion 113 forms a channel 115. The channel 115 extends from one of the inlet 131 and the outlet 132 of the fluidic chamber 130 to the apex of the protrusion 114. For example, in the embodiment shown in FIG. 1, the channel 115 extends from the inlet 131 to the apex of the protrusion 114. However, in alternative embodiments are discussed in further detail below with regard to FIGS. 5-6, the channel 115 may extend from the outlet 132 to the apex of the protrusion 114.

As noted above, the volume of the fluidic chamber 130 is bounded by the first surface 111 of the first piece 110 and the second surface 121 of the second piece 120. Because the protrusion 113 is included in the first piece 110 and is bounded by the first surface 111 of the first piece 110, the protrusion 113 in part defines the volume of the fluidic chamber 130. In some embodiments, the fluidic chamber 130 is a microfluidic chamber. For example, in certain embodiments, the volume of the fluidic chamber 130 can be between 1 μL to 1100 μL. In a further embodiment, the volume of the fluidic chamber 130 can be on the order of 30 μL.

The protrusion 113 also in part defines a shape of the volume of the fluidic chamber 130. Specifically, the protrusion 113 is shaped such that when the first piece 110 and the second piece 120 are operatively coupled and the protrusion 113 protrudes into the fluidic chamber 130, a cross-sectional area of the volume of the fluidic chamber 130 increases from the apex of the protrusion 114, where the cross-sectional area is defined in part by the distance of minimal approach, to the transverse plane of the fluidic chamber 130, and then decreases from the transverse plane of the fluidic chamber 130 to the other one of the one of the inlet 131 and the outlet 132 from which the channel 115 extends. In such embodiments in which the cross-sectional area of the volume of the fluidic chamber 130 increases from the apex of the protrusion 114 to the transverse plane and decreases from the transverse plane to the other one of the one of the inlet 131 and the outlet 132 from which the channel 115 extends, apart from the channel 115, the volume of the fluidic chamber 130 is substantially shaped as a quadrilateral prism, as shown in FIG. 1. In alternative embodiments, the volume of the fluidic chamber 130 can comprise any other shape, for example a cylinder, rectangular box, cube, or any combination thereof.

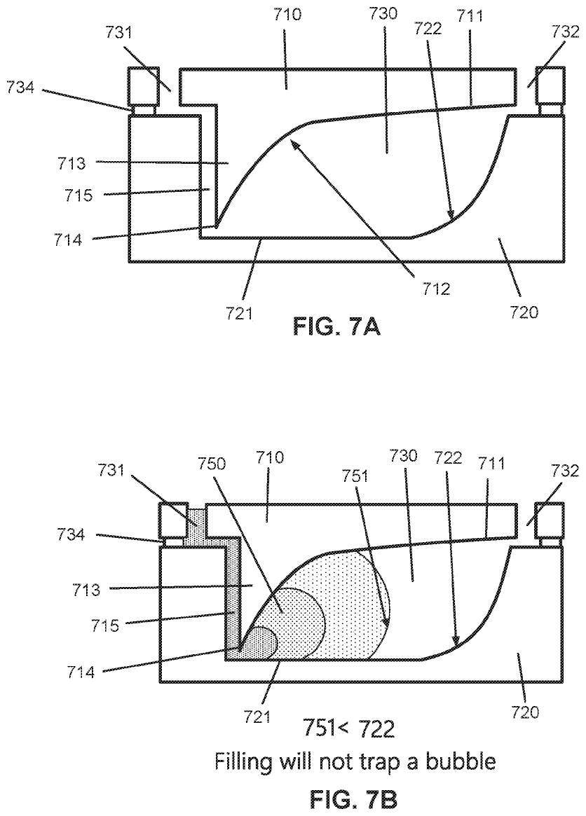

As described in detail below with regard to FIGS. 7A-B, the shape of the volume of the fluidic chamber 130, as defined by the protrusion 113, aids in avoidance of bubble formation during filling of the fluidic chamber 130 with a liquid in multiple ways. First, the protrusion 113, and the channel 115 formed by the protrusion 113, enables the inlet 131 and the outlet 132 to be separated from one another as much as possible, such that a maximum distance of travel through the volume of the fluidic chamber 130 exists between the inlet 131 and the outlet 132. Specifically, positioning the protrusion 113, and thus the channel 115, between the inlet 131 and the outlet 132, increases the distance of travel through the volume of the fluidic chamber 130 between the inlet 131 and the outlet 132. Additionally, in certain embodiments, such as the embodiment shown in FIG. 1, forming both the inlet 131 and the outlet 132 in the first piece 110 of the assembly 100, such that the apex of the protrusion 114 is located diagonally across the volume of the fluidic chamber 130 from the inlet 131 or the outlet 132, further maximizes the separation between the inlet 131 and the outlet 132. This maximum possible separation between the inlet 131 and the outlet 132 of the fluidic chamber aids in avoiding bubble formation as the fluidic chamber 130 fills with liquid because . . . .

Second, the cross-sectional area of the volume of the fluidic chamber 130 increasing from the apex of the protrusion 114 to the transverse plane, and decreasing from the transverse plane to the other one of the one of the inlet 131 and the outlet 132 of the fluidic chamber 130 from which the channel 115 extends, enables a liquid to gradually fill the fluidic chamber 130 between the apex of the protrusion 114 and the other one of the one of the inlet 131 and the outlet 132, thereby further aiding in avoidance of bubble formation during filling of the fluidic chamber 130 with the liquid. Specifically, the cross-sectional area of the volume of the fluidic chamber 130 increasing from the apex of the protrusion 114 to the transverse plane, and decreasing from the transverse plane to the other one of the one of the inlet 131 and the outlet 132 of the fluidic chamber 130 from which the channel 115 extends, enables a liquid to gradually fill the volume of the fluidic chamber 130 such that a radius of curvature of a meniscus of the liquid increases from the apex of the protrusion 114 to the transverse plane of the fluidic chamber 130, and decreases from the transverse plane of the fluidic chamber 130 to the other one of the one of the inlet 131 and the outlet 132 of the fluidic chamber 130, but does not surpass a radius of curvature of the surfaces of the fluidic chamber 130. As discussed further below with regard to FIGS. 7A-B, this minimization of the radius of the liquid filling the fluidic chamber 130 relative to the radii of curvature of the surfaces of the fluidic chamber 130, as enabled by the shape of the fluidic chamber 130, minimizes the trapping of bubbles within the fluidic chamber 130 during filling.

In some embodiments, the first surface 111 of the first piece 110 and the second surface 121 of the second piece 120 have a roughness value of less than 25 micro-inches to further prevent formation and catching of bubbles along the surfaces of the fluidic chamber 130.

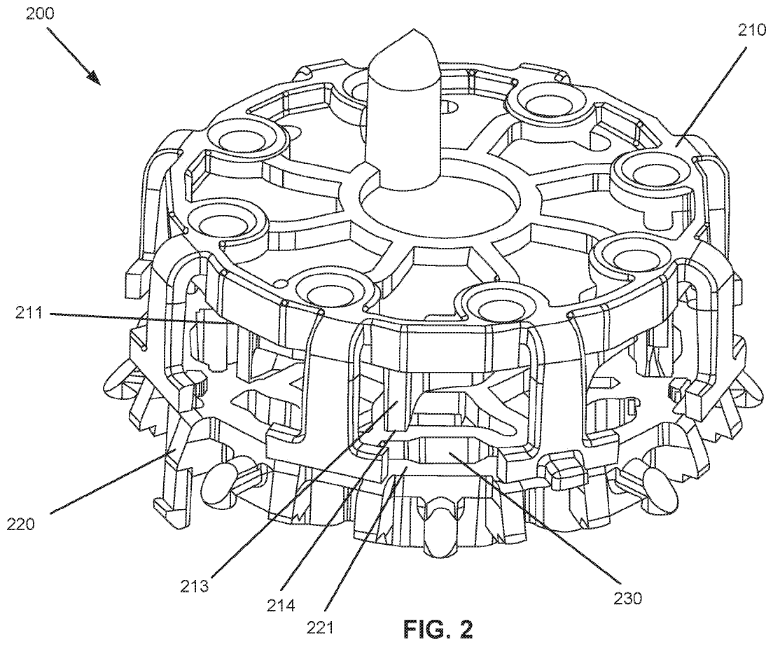

FIG. 2 is a diagram of an assembly 200 for avoiding bubble formation in a fluidic chamber 230 of the assembly, during filling of the fluidic chamber 230 with a liquid, in accordance with an embodiment. The assembly 200 of FIG. 2 is similar to the assembly 100 of FIG. 1. However, unlike the assembly 100 of FIG. 1, a first piece 210 and a second piece 220 of the assembly 200 of FIG. 2 are uncoupled for visualization purposes. As shown in FIG. 2, the first piece 110 comprises a protrusion 213 that is configured to protrude into the fluidic chamber 230, thereby defining a volume and shape of the fluidic chamber 230, when the first piece 210 and the second piece 220 are operatively coupled to one another.

Additionally, as shown in the embodiment in FIG. 2, the operative coupling of the first piece 210 and the second piece 220 of the assembly 200 not only forms the single fluidic chamber 230, but forms a plurality of fluidic chambers. In such embodiments, the volume of each fluidic chamber of the plurality of fluidic chambers may be the same, or alternatively, the volume of at least one of the plurality of fluidic chambers may differ from the volume of at least one other of the plurality of fluidic chambers. Furthermore, in some embodiments, each fluidic chamber of the plurality of fluidic chambers may be independent of the other fluidic chambers. Alternatively, each fluidic chamber of the plurality of fluidic chambers may be in fluidic communication with at least one other fluidic chamber of the plurality of fluidic chambers. Fluidic communication between a first fluidic chamber and a second fluidic chamber may be achieved by the presence of a fluidic connection between one of an inlet and an outlet of the first fluidic chamber and the other of the one of the inlet and the outlet of the second fluidic chamber. For example, a first fluidic chamber and a second fluidic chamber may be in fluidic communication with one another via a fluidic connection between the outlet of the first fluidic chamber and the inlet of the second fluidic chamber. As a further example, the second fluidic chamber may also be in fluidic communication with a third fluidic chamber via a fluidic connection between the outlet of the second fluidic chamber and the inlet of the third fluidic chamber.

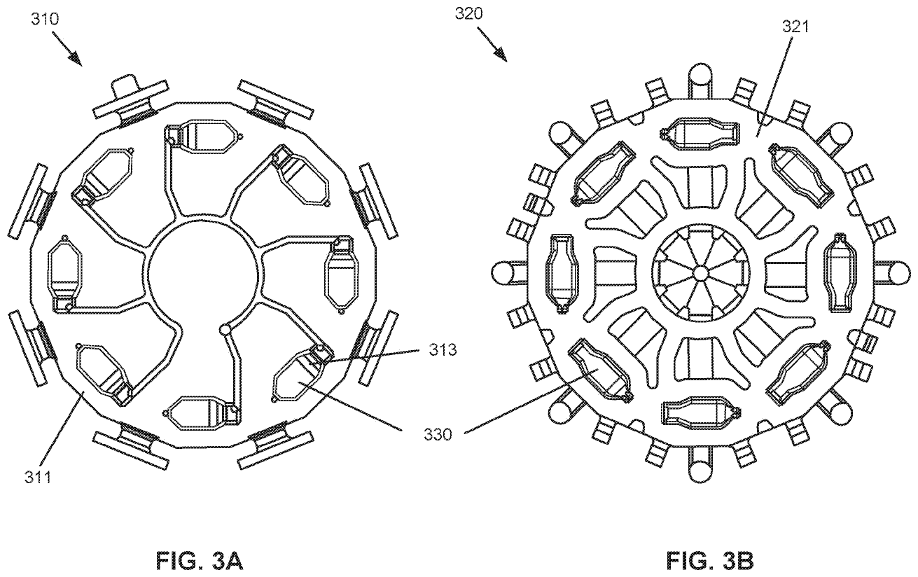

FIG. 3A is a diagram of a first surface 311 of a first piece 310 of an assembly for avoiding bubble formation in a fluidic chamber 330 of the assembly, during filling of the fluidic chamber 330 with a liquid, in accordance with an embodiment. Similarly, FIG. 3B is a diagram of a second surface 321 of a second piece 320 of an assembly for avoiding bubble formation in the fluidic chamber 330 of the assembly, during filling of the fluidic chamber 330 with a liquid, in accordance with an embodiment. Like the assembly 200 of FIG. 2, the first piece 310 and the second piece 320 of FIGS. 3A-B are uncoupled for visualization purposes. As described above, when the first piece 310 and the second piece 320 are operatively coupled to one another, the fluidic chamber 330 is formed, and the volume of the fluidic chamber 330 is bounded by the first surface 311 of the first piece 310 and the second surface 321 of the second piece 320.

Like the assembly 200 of FIG. 2, in the embodiment of the assembly depicted in FIGS. 3A and 3B, the operative coupling of the first piece 310 and the second piece 320 not only forms the single fluidic chamber 330, but forms a plurality of fluidic chambers. In the embodiment of the assembly depicted in FIGS. 3A and 3B, each fluidic chamber 330 of the plurality of fluidic chambers is independent of the other fluidic chambers. More specifically, in the embodiment of the assembly depicted in FIGS. 3A and 3B, once liquid enters a fluidic chamber it cannot exit the fluidic chamber to enter another fluidic chamber. (The channels connecting the fluidic chambers in FIG. 3A are configured to supply a liquid to each fluidic chamber from a common source, but not to fluidically connect the fluidic chambers after the liquid has entered the fluidic chambers.) However, in alternative embodiments, each fluidic chamber of the plurality of fluidic chambers may be in fluidic communication with at least one other fluidic chamber of the plurality of fluidic chambers such that liquid can travel from one fluidic chamber into another fluidic chamber. For example, in alternative embodiments, a first fluidic chamber and a second fluidic chamber may be in fluidic communication with one another via a fluidic connection between the outlet of the first fluidic chamber and the inlet of the second fluidic chamber. As a further example, the second fluidic chamber may also be in fluidic communication with a third fluidic chamber via a fluidic connection between the outlet of the second fluidic chamber and the inlet of the third fluidic chamber.

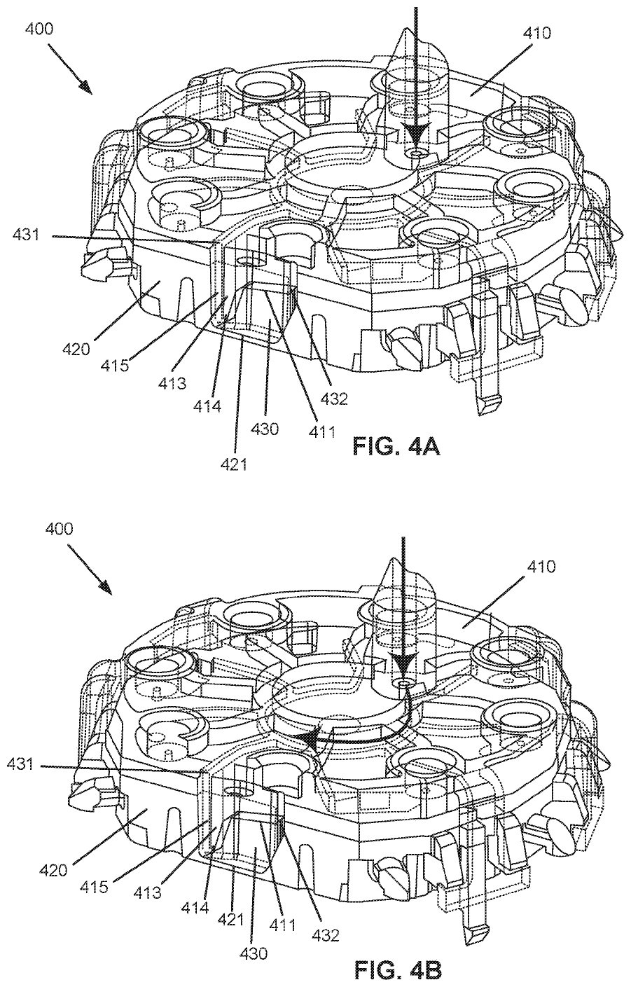

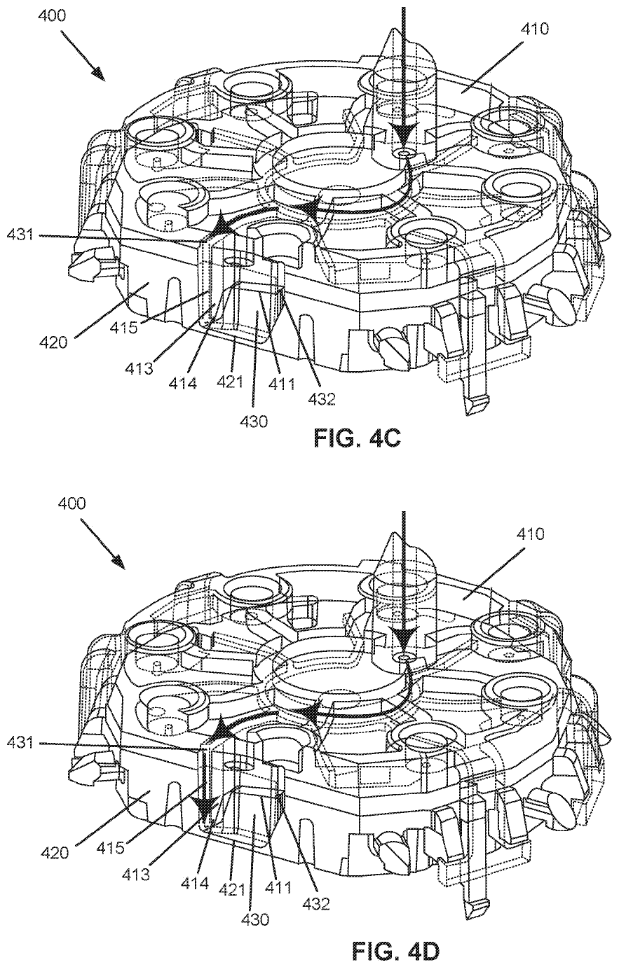

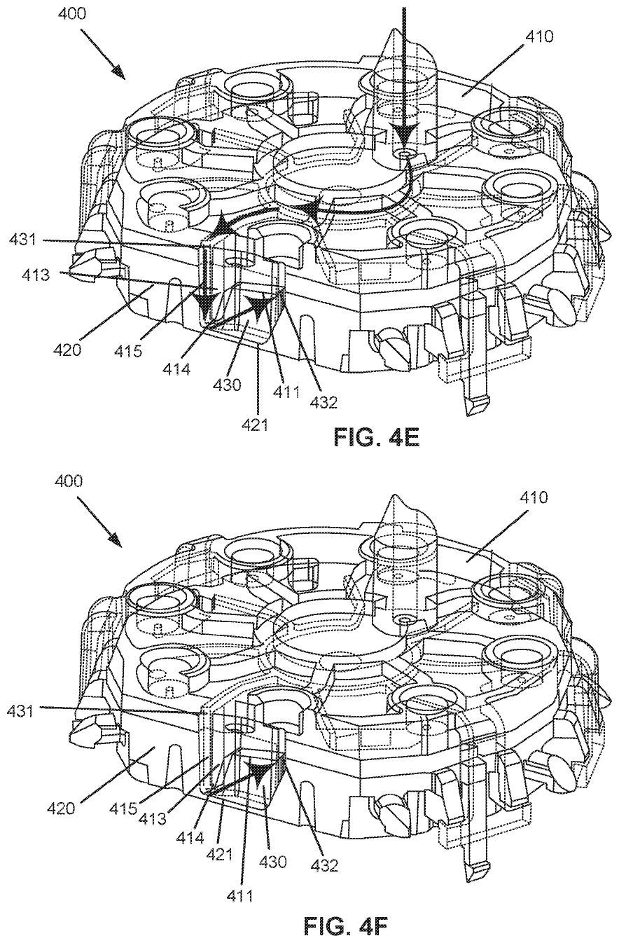

FIGS. 4A-F depict an assembly 400 at a plurality of sequential time points during filling of a fluidic chamber 430 of the assembly 400 with a liquid, in accordance with an embodiment. The flow of the liquid is denoted in FIGS. 4A-F by arrows.

As seen in FIGS. 4A-F, the assembly 400 comprises a first piece 410 operatively coupled to a second piece 420 to form the fluidic chamber 430. The fluidic chamber 430 includes an inlet 431 and an outlet 432. The first piece 410 of the assembly 400 includes a protrusion 413 that protrudes into the fluidic chamber 430 such that there is a distance of minimal approach between an apex of the protrusion 414 and the second surface 421 of the second piece 420. The protrusion 413 also forms a channel 415 that extends from the inlet 431 to the apex of the protrusion 414. In alternative embodiments discussed in further detail with regard to FIGS. 5-6, the protrusion 415 may be positioned differently within the fluidic chamber 430 such that the channel 413 extends from the outlet 432 to the apex of the protrusion 414.

As shown in FIGS. 4A-F, a maximum possible distance of travel through the volume of the fluidic chamber 430 exists between the inlet 431 and the outlet 432. This maximal separation of the inlet 431 and the outlet 432 is accomplished by positioning the protrusion 413, and thus the channel 415, between the inlet 431 and the outlet 432, and by the inlet 431 and the outlet 432 both being formed in the first piece 410 of the assembly 400 such that the apex of the protrusion 414 is located diagonally across the volume of the fluidic chamber 430 from the outlet 432. Additionally, a cross-sectional area of the volume of the fluidic chamber 430 increases from the apex of the protrusion 414 to a transverse plane, and decreases from the transverse plane to the outlet 432. This increasing cross-sectional area of the volume of the fluidic chamber 430 from the apex of the protrusion 414 to the transverse plane is accomplished in part by the distance of minimal approach between the apex of the protrusion 414 and the second surface 421 of the second piece 420 being less than a largest dimension of the cross-sectional area of the volume of the fluidic chamber 430 at the transverse plane of the fluidic chamber 430.

Turning to FIGS. 4A-C, FIGS. 4A-C depict the assembly 400 at times A-C, respectively, during filling of the fluidic chamber 430 of the assembly 400 with a liquid, in accordance with an embodiment. In particular, FIGS. 4A-C depict the liquid flowing through the first piece 410 of the assembly 400 until the liquid reaches the inlet 431 of the fluidic chamber 430.

FIG. 4D depicts the assembly 400 at a time D, during filling of the fluidic chamber 430 of the assembly 400 with a liquid, in accordance with an embodiment. In particular, FIG. 4D depicts the liquid flowing from the inlet 431 of the fluidic chamber 430, through the channel 415, and towards the apex of the protrusion 414.

FIG. 4E depicts the assembly 400 at a time E, during filling of the fluidic chamber 430 of the assembly 400 with a liquid, in accordance with an embodiment. In particular, FIG. 4E depicts the liquid flowing from the distance of minimal approach between the apex 414 and the second surface 421 of the second piece 420, towards the outlet 432 of the fluidic chamber 430. Due to the maximum possible distance of travel through the volume of the fluidic chamber 430 between the inlet 431 and the outlet 432, and the cross-sectional area of the volume of the fluidic chamber 430 increasing from the apex of the protrusion 414 to the transverse plane and then decreasing from the transverse plane to the outlet 432, a radius of curvature of a meniscus of the liquid filling the fluidic chamber 430 is prevented from surpassing radii of curvature of surfaces of the fluidic chamber 430, thereby minimizing bubble formation during filling of the fluidic chamber 430 with the liquid.

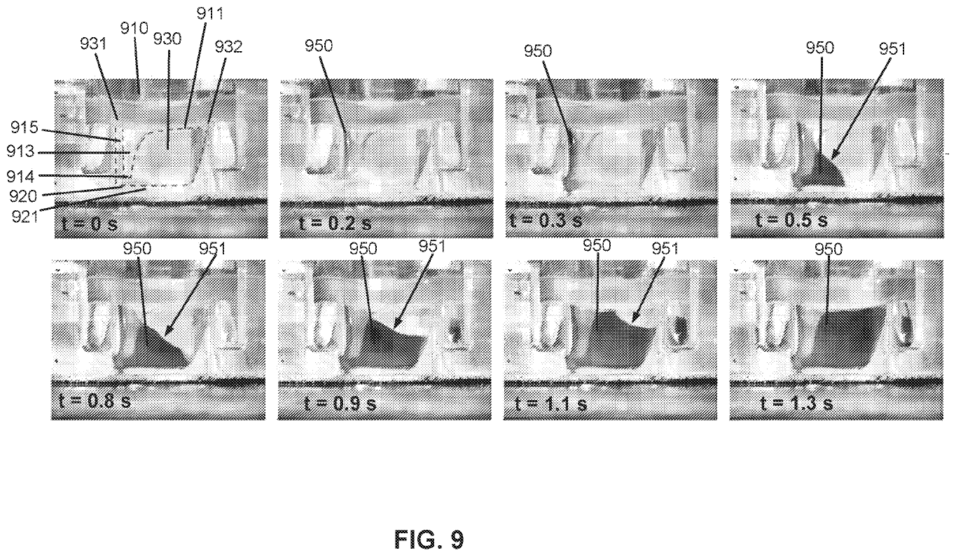

FIG. 4F depicts the assembly 400 at a time F, during filling of the fluidic chamber 430 of the assembly 400 with a liquid, in accordance with an embodiment. In particular, FIG. 4F depicts a final stage of the flow of the liquid through the assembly 400. In FIG. 4F, all liquid is contained in the volume of the fluidic chamber 430, the liquid having filled the fluidic chamber 430 without the formation of bubbles. A sequence of time-lapse images depicting filling of a fluidic chamber of a working embodiment of the assembly of FIGS. 4A-F is depicted in FIG. 9 and described in detail below.

### Fluidic Chambers

FIGS. 5A-F depict multiple embodiments of a fluidic chamber 530 configured to avoid bubble formation during filling of the fluidic chamber 530 with a liquid. Each of the embodiments of the fluidic chamber 530 of FIGS. 5A-F varies according to one or more of: orientation of the fluidic chamber 530 with respect to the force of gravity, quantity of protrusions and channels of the fluidic chamber 530, and positioning of channels of the fluidic chamber 530 with respect to an inlet and an outlet of the fluidic chamber 530. The direction of gravity is indicated at the top of the set of FIGS. 5A-F. Each of the embodiments of the fluidic chamber 530 of FIGS. 5A-F is discussed in detail below.

Turning first to the embodiment of the fluidic chamber depicted in FIG. 5A, FIG. 5A depicts a first fluidic chamber 530, in accordance with an embodiment. The fluidic chamber 530 is formed by the operative coupling of a first piece 510 and a second piece 520. In the embodiment shown in FIG. 5A, the first piece 510 and the second piece 520 are operatively coupled by a gasket 534. A first surface 511 of the first piece 510 and a second surface 521 of the second piece 520 bound a volume of the fluidic chamber 530. The fluidic chamber 530 includes an inlet 531 and an outlet 532.

The first piece 510 includes a protrusion 513 that is bounded by the first surface 511 of the first piece 510. The protrusion 513 protrudes into the fluidic chamber 530 such that there is a distance of minimal approach between an apex of the protrusion 514 and the second surface 521 of the second piece 520. In the embodiment depicted in FIG. 5A, the distance of minimal approach between the apex of the protrusion 514 and the second surface 521 of the second piece 520 is less than a largest dimension of the cross-sectional area of the volume of the fluidic chamber 530 at a transverse plane of the fluidic chamber 530.

The protrusion 513 forms a channel 515 that extends from the outlet 532 of the fluidic chamber 530 to the apex of the protrusion 514. Both the inlet 531 and the outlet 532 of the fluidic chamber 530 are formed in the first piece 510 of the fluidic chamber 530 such that the apex of the protrusion 514 is located diagonally across the volume of the fluidic chamber 530 from the inlet 531, and such that a maximum distance of travel through the volume of the fluidic chamber 530 exists between the inlet 531 and the outlet 532.

A cross-sectional area of the volume of the fluidic chamber 530 increases from the apex of the protrusion 514, where the cross-sectional area is defined in part by the distance of minimal approach, to the transverse plane of the fluidic chamber 530, and decreases from the transverse plane of the fluidic chamber 530 to the inlet 531 of the fluidic chamber 530.

As shown in FIG. 5A, the fluidic chamber 530 is oriented with respect to gravity such that the second piece 520 of the fluidic chamber 530 is located in the direction of the force of gravity. In this orientation, as well as in any other orientation (as discussed in further detail below with regard to FIG. 5C), the fluidic chamber 530 of FIG. 5A is able to avoid bubble formation during filling of the fluidic chamber 530 with a liquid.

Turning next to the embodiment of the fluidic chamber depicted in FIG. 5B, FIG. 5B depicts a second fluidic chamber 530, in accordance with an embodiment. The fluidic chamber 530 of FIG. 5B is similar to the fluidic chamber of FIG. 5A. However, unlike the fluidic chamber of FIG. 5A, the first piece 510 of the fluidic chamber 530 of FIG. 5B includes a protrusion 513 that forms a channel 515 that extends from an inlet 531 of the fluidic chamber 530 to an apex of the protrusion 514.

Both the inlet 531 and outlet 532 of the fluidic chamber 530 are formed in the first piece 510 of the fluidic chamber 530 such that the apex of the protrusion 514 is located diagonally across the volume of the fluidic chamber 530 from the outlet 532, and such that a maximum distance of travel through the volume of the fluidic chamber 530 exists between the inlet 531 and the outlet 532.

A cross-sectional area of the volume of the fluidic chamber 530 increases from the apex of the protrusion 514, where the distance comprises a distance of minimal approach, to a transverse plane of the fluidic chamber 530, and decreases from the transverse plane of the fluidic chamber 530 to the outlet 532 of the fluidic chamber 530.

As shown in FIG. 5B, the fluidic chamber 530 is oriented with respect to gravity such that the second piece 520 of the fluidic chamber 530 is located in the direction of the force of gravity. In this orientation, as well as in any other orientation (as discussed in further detail below with regard to FIG. 5D), the fluidic chamber 530 of FIG. 5B is able to avoid bubble formation during filling of the fluidic chamber 530 with a liquid.

Turning next to the embodiment of the fluidic chamber depicted in FIG. 5C, FIG. 5C depicts a third fluidic chamber 530, in accordance with an embodiment. The fluidic chamber 530 of FIG. 5C is identical to the fluidic chamber of FIG. 5A. However, unlike the fluidic chamber of FIG. 5A, the fluidic chamber 530 of FIG. 5C is oriented with respect to gravity such that a first piece 510 of the fluidic chamber 530 is located in the direction of the force of gravity. Despite this flipped orientation of the fluidic chamber 530 of FIG. 5C, the fluidic chamber 530 of FIG. 5C is still able to avoid bubble formation during filling of the fluidic chamber 530 with a liquid. In other words, the fluidic chamber 530 of FIGS. 5A and 5C is configured to avoid bubble formation during filling of the fluidic chamber 530 both when the fluidic chamber 530 is oriented such that the first piece 510 of the fluidic chamber 530 is located in the direction of the force of gravity, and when the fluidic chamber 530 is oriented such that the second piece 520 of the fluidic chamber 530 is located in the direction of the force of gravity. Even further, in addition to the orientations depicted in FIGS. 5A and 5C, the fluidic chamber 530 FIGS. 5A and 5C is configured to avoid bubble formation during filling of the fluidic chamber 530 in any orientation. And as discussed with regard to additional examples below, this ability to avoid bubble formation during filling in any orientation holds true not only for the fluidic chamber 530 of FIGS. 5A and 5C, but for any embodiment of the fluidic chambers disclosed herein.

Turning next to the embodiment of the fluidic chamber depicted in FIG. 5D, FIG. 5D depicts a fourth fluidic chamber 530, in accordance with an embodiment. The fluidic chamber 530 of FIG. 5D is identical to the fluidic chamber of FIG. 5B. However, unlike the fluidic chamber of FIG. 5B, the fluidic chamber 530 of FIG. 5D is oriented with respect to gravity such that a first piece 510 of the fluidic chamber 530 is located in the direction of the force of gravity. Despite this flipped orientation of the fluidic chamber 530 of FIG. 5D, the fluidic chamber 530 of FIG. 5D is still able to avoid bubble formation during filling of the fluidic chamber 530 with a liquid. In other words, the fluidic chamber 530 of FIGS. 5B and 5D is configured to avoid bubble formation during filling of the fluidic chamber 530 both when the fluidic chamber 530 is oriented such that the first piece 510 of the fluidic chamber 530 is located in the direction of the force of gravity, and when the fluidic chamber 530 is oriented such that the second piece 520 of the fluidic chamber 530 is located in the direction of the force of gravity. Even further, in addition to the orientations depicted in FIGS. 5B and 5D, the fluidic chamber 530 FIGS. 5B and 5D is configured to avoid bubble formation during filling of the fluidic chamber 530 in any orientation. And as discussed above, this ability to avoid bubble formation during filling in any orientation holds true not only for the fluidic chamber 530 of FIGS. 5B and 5D, but for any embodiment of the fluidic chambers disclosed herein.

Turning next to the embodiment of the fluidic chamber depicted in FIG. 5E, FIG. 5E depicts a fifth fluidic chamber 530, in accordance with an embodiment. Unlike the embodiments of the fluidic chamber depicted in FIGS. 5A-D, the fluidic chamber 530 depicted in FIG. 5E includes two protrusions and two channels, each channel formed by one of the two protrusions.

The fluidic chamber 530 of FIG. 5E is formed by the operative coupling of a first piece 510 and a second piece 520. A first surface 511 of the first piece 510 and a second surface 521 of the second piece 520 bound a volume of the fluidic chamber 530. The fluidic chamber 530 includes an inlet 531 and an outlet 532.

The first piece 510 includes a protrusion 513 that is bounded by the first surface 511 of the first piece 510. The protrusion 513 protrudes into the fluidic chamber 530 such that there is a distance of minimal approach between an apex of the protrusion 514 and the second surface 521 of the second piece 520. In the embodiment depicted in FIG. 5E, the distance of minimal approach between the apex of the protrusion 514 and the second surface 521 of the second piece 520 is less than a largest dimension of the cross-sectional area of the volume of the fluidic chamber 530 at the transverse plane of the fluidic chamber 530. The protrusion 513 forms a channel 515 that extends from the inlet 531 of the fluidic chamber 530 to the apex of the protrusion 514.

In addition to the protrusion 513 included in the first piece 510, the second piece 520 also includes a second protrusion 523. The second protrusion 523 is bounded by the second surface 521 of the second piece 520. The second protrusion 523 protrudes into the fluidic chamber 530 such that there is a second distance of minimal approach between an apex of the second protrusion 524 and the first surface 511 of the first piece 510. In the embodiment depicted in FIG. 5E, the second distance of minimal approach between the apex of the second protrusion 524 and the first surface 511 of the first piece 510 is less than a largest dimension of the cross-sectional area of the volume of the fluidic chamber 530 at the transverse plane of the fluidic chamber 530. The second protrusion 523 forms a second channel 525 that extends from the outlet 532 of the fluidic chamber 530 to the apex of the second protrusion 524.

As shown in FIG. 5E, the inlet 531 of the fluidic chamber 530 is formed in the first piece 510 of the fluidic chamber 530, and the outlet 532 of the fluidic chamber 530 is formed in the second piece 520 of the fluidic chamber 530, such that the apex of the second protrusion 524 is located diagonally across the volume of the fluidic chamber 530 from the apex of the protrusion 514, such that the inlet 531 of the fluidic chamber 530 is located diagonally across the volume of the fluidic chamber 530 from the outlet 532 of the fluidic chamber, and such that a maximum distance of travel through the volume of the fluidic chamber 530 exists between the inlet 531 and the outlet 532.

As discussed above, the volume of the fluidic chamber 530 is bounded by the first surface 511 of the first piece 510 and the second surface 521 of the second piece 520. Because the protrusion 513 is included in the first piece 510 and is bounded by the first surface 511 of the first piece 510, and the second protrusion 523 is included in the second piece 520 and is bounded by the second surface 521 of the second piece 520, the protrusion 513 and the second protrusion 523 in part define the volume of the fluidic chamber 530. As with embodiments in which a fluidic chamber comprises only a single protrusion, in some embodiments, the fluidic chamber 530 is a microfluidic chamber. For example, in certain embodiments, the volume of the fluidic chamber 530 can be between 1 μL to 1100 μL. In a further embodiment, the volume of the fluidic chamber 530 can be on the order of 30 μL.

The protrusions 513 and 523 also define a shape of the volume of the fluidic chamber 530. Specifically, the protrusion 513 is shaped such that when the first piece 510 and the second piece 520 are operatively coupled, and the protrusion 513 protrudes into the fluidic chamber 530, a cross-sectional area of the volume of the fluidic chamber 530 increases from the apex of the protrusion 514, where the cross-sectional area is defined in part by the distance of minimal approach, to the transverse plane of the fluidic chamber 530. And furthermore, the second protrusion 523 is shaped such that when the first piece 510 and the second piece 520 are operatively coupled, and the protrusion 523 protrudes into the fluidic chamber 530, the cross-sectional area of the volume of the fluidic chamber 530 decreases from the transverse plane of the fluidic chamber 530 to the apex of the second protrusion 524 of the fluidic chamber 530, where the cross-sectional area is defined in part by the second distance of minimal approach. In such embodiments in which the cross-sectional area of the volume of the fluidic chamber 530 increases from the apex of the protrusion 514 to the transverse plane and decreases from the transverse plane to the apex of the second protrusion 524, apart from the channel 115 and the channel 125, the volume of the fluidic chamber 530 is substantially shaped as a quadrilateral prism. In alternative embodiments, the volume of the fluidic chamber 530 can comprise any other shape, for example a cylinder, rectangular box, cube, or any combination thereof.

As described in detail below with regard to FIGS. 7A-B, the shape of the volume of the fluidic chamber 530, as defined in part by the protrusion 513 and the second protrusion 523, aids in avoidance of bubble formation during filling of the fluidic chamber 530 with a liquid in multiple ways. First, the protrusions 513 and 523, and the channels 515 and 525 respectively formed by the protrusions 513 and 523, enable the inlet 531 and the outlet 532 to be separated from one another as much as possible, such that a maximum distance of travel through the volume of the fluidic chamber 530 exists between the inlet 531 and the outlet 532. Specifically, the positioning of the protrusions 513 and 523, and thus the channels 515 and 525, between the inlet 531 and the outlet 532, increases the distance of travel through the volume of the fluidic chamber 530 between the inlet 531 and the outlet 532. Additionally, forming the inlet 531 and the outlet 532 in opposing pieces (e.g., the first piece 510 and the second piece 520) of the fluidic chamber 530, such that the apex of the second protrusion 524 is located diagonally across the volume of the fluidic chamber 530 from the apex of the protrusion 514, and such that the inlet 531 of the fluidic chamber 530 is located diagonally across the volume of the fluidic chamber 530 from the outlet 532 of the fluidic chamber, further maximizes the separation between the inlet 531 and the outlet 532. This maximum possible separation between the inlet 531 and the outlet 532 of the fluidic chamber aids in avoiding bubble formation as the fluidic chamber 530 fills with liquid because . . . .

Second, the cross-sectional area of the volume of the fluidic chamber 530 increasing from the apex of the protrusion 514 to the transverse plane, and decreasing from the transverse plane to the apex of the second protrusion 524, enables a liquid to gradually fill the fluidic chamber 530 between the apex of the protrusion 514 and the apex of the second protrusion 524, thereby further aiding in avoidance of bubble formation during filling of the fluidic chamber 530 with the liquid. Specifically, the cross-sectional area of the volume of the fluidic chamber 530 increasing from the apex of the protrusion 514 to the transverse plane, and decreasing from the transverse plane to the apex of the second protrusion 524, enables a liquid to gradually fill the volume of the fluidic chamber 530 such that a radius of curvature of a meniscus of the liquid increases from the apex of the protrusion 514 to the transverse plane of the fluidic chamber 530, and decreases from the transverse plane of the fluidic chamber 530 to the apex of the second protrusion 524 of the fluidic chamber 530, but does not surpass radii of curvature of the surfaces of the fluidic chamber 530. As discussed further below with regard to FIGS. 7A-B, this minimization of the radius of curvature of the meniscus of the liquid filling the fluidic chamber 530 relative to the radii of curvature of the surfaces of the fluidic chamber 530, as enabled by the shape of the fluidic chamber 530, minimizes the trapping of bubbles within the fluidic chamber 530 during filling.

As shown in FIG. 5E, the fluidic chamber 530 is oriented with respect to gravity such that the second piece 520 of the fluidic chamber 530 is located in the direction of the force of gravity. In this orientation with respect to gravity, as well as in any other orientation with respect to gravity (as discussed in further detail below with regard to FIG. 5F), the fluidic chamber 530 of FIG. 5E is able to avoid bubble formation during filling of the fluidic chamber 530 with a liquid.

Turning finally to the embodiment of the fluidic chamber depicted in FIG. 5F, FIG. 5F depicts a sixth fluidic chamber 530, in accordance with an embodiment. The fluidic chamber 530 of FIG. 5F is identical to the fluidic chamber of FIG. 5E. However, unlike the fluidic chamber of FIG. 5E, the fluidic chamber 530 of FIG. 5F is oriented with respect to gravity such that a first piece 510 of the fluidic chamber 530 is located in the direction of the force of gravity. Despite this flipped orientation of the fluidic chamber 530 of FIG. 5F, the fluidic chamber 530 of FIG. 5F is still able to avoid bubble formation during filling of the fluidic chamber 530 with a liquid. In other words, the fluidic chamber 530 of FIGS. 5E and 5F is configured to avoid bubble formation during filling of the fluidic chamber 530 both when the fluidic chamber 530 is oriented such that the first piece 510 of the fluidic chamber 530 is located in the direction of the force of gravity, and when the fluidic chamber 530 is oriented such that the second piece 520 of the fluidic chamber 530 is located in the direction of the force of gravity. Even further, in addition to the orientations depicted in FIGS. 5E and 5F, the fluidic chamber 530 FIGS. 5E and 5F is configured to avoid bubble formation during filling of the fluidic chamber 530 in any orientation. This ability to avoid bubble formation during filling in any orientation holds true for any embodiment of the fluidic chambers disclosed herein.

Despite the bubble-preventing features of the fluidic chambers described herein, in some embodiments, bubbles may form during filling of a fluidic chamber. Additionally, in certain embodiments, after a fluidic chamber has been filled with a liquid, an assay may be executed within the fluidic chamber causing formation of bubbles within the fluidic chamber. As discussed throughout this disclosure, these bubbles may interfere with execution of an assay itself and/or with collection of assay results. For example, bubbles may interfere with detection of optical properties of the assay. Therefore, in addition to configuring a fluidic chamber to avoid bubble formation, in some embodiments it may also be beneficial to configure the fluidic chamber to remove and/or displace bubbles within the fluidic chamber. Such embodiments are depicted in FIGS. 6A-F.

FIGS. 6A-F depict multiple embodiments of a fluidic chamber 630 configured to not only avoid bubble formation during filling of the fluidic chamber 630 with a liquid, but to and/or displace bubbles within the fluidic chamber 630. The embodiments of the fluidic chamber 630 of FIGS. 6A-F are similar to the embodiments of the fluidic chamber 530 of FIGS. 5A-F. However, unlike the embodiments of the fluidic chamber 530 of FIGS. 5A-F, at a surface (e.g., a first surface or a second surface) of each embodiment of the fluidic chamber 630 of FIGS. 6A-F includes a sloping point. As discussed in further detail below, a sloping point of a surface of the fluidic chamber 630 denotes a location along the surface of the fluidic chamber 630 at which the surface begins to slope away from the other surface of the fluidic chamber 630. Removal of bubbles from the fluidic chamber 630 via the sloping surface is contingent on the orientation of the fluidic chamber 630 with respect to gravity. Specifically, removal of bubbles from the fluidic chamber 630 via the sloping surface is contingent on the sloping surface being located in the direction opposite the force of gravity with respect to the other surface of the fluidic chamber 630. The direction of gravity is indicated at the top of the set of FIGS. 6A-F. Contingent on this orientation of the fluidic chamber 630, due to buoyant forces, bubbles can rise in the fluidic chamber 630 towards the sloping surface, and then travel along the sloping surface of the fluidic chamber 630 in a direction opposite the direction of the force of gravity and towards one of an inlet, an outlet, or an apex of a protrusion of the fluidic chamber 630, where the bubble can escape the fluidic chamber 630. Each of the embodiments of the fluidic chamber 630 of FIGS. 6A-F is discussed in detail below.

Turning first to the embodiment of the fluidic chamber depicted in FIG. 6A, FIG. 6A depicts a first fluidic chamber 630, in accordance with an embodiment. The fluidic chamber 630 of FIG. 6A is similar to the fluidic chambers 530 of FIGS. 5A and 5C. However, unlike the fluidic chambers 530 of FIGS. 5A and 5C, a first surface 611 of the fluidic chamber 630 of FIG. 6A includes a sloping point 616. As shown in FIG. 6A, the first surface 611 slopes away from a second surface 621 of the fluidic chamber 630 from the sloping point 616 towards an inlet 631 of the fluidic chamber 630.

As discussed above, removal of bubbles from the fluidic chamber 630 via a sloping surface is contingent on the orientation of the fluidic chamber 630 with respect to gravity. Specifically, removal of bubbles from the fluidic chamber 630 via the sloping surface is contingent on the sloping surface being located in the direction opposite the force of gravity with respect to the other surface of the fluidic chamber 630. Thus, the fluidic chamber 630 of FIG. 6A is oriented with respect to gravity such that the first surface 611 that includes the sloping point 616 is located in the direction opposite the force of gravity with respect to the second surface 621 of the fluidic chamber 630. In this orientation, bubbles formed within the fluidic chamber 630 are able to rise in the fluidic chamber 630 towards the first surface 611 and then travel along the first surface 611 of the fluidic chamber 630 towards the inlet 631 of the fluidic chamber 630 in a direction opposite the direction of the force of gravity, due to buoyant forces. In some embodiments, once the bubbles reach the inlet 631 of the fluidic chamber 630, the bubbles exit the fluidic chamber 630 via the inlet 631. Alternatively, the bubbles may remain within the fluidic chamber 630 along the first surface 611, but are displaced from the center of the volume of the fluidic chamber 630 such that they do not interfere, for example, with execution of an assay and/or with collection of assay results. An embodiment of the fluidic chamber 630 in which the second surface 621, rather than the first surface 611, of the fluidic chamber 630 includes a sloping point is discussed in detail below with regard to FIG. 6C.

Turning next to the embodiment of the fluidic chamber depicted in FIG. 6B, FIG. 6B depicts a second fluidic chamber 630, in accordance with an embodiment. The fluidic chamber 630 of FIG. 6B is similar to the fluidic chambers 530 of FIGS. 5B and 5D. However, unlike the fluidic chambers 530 of FIGS. 5B and 5D, a first surface 611 of the fluidic chamber 630 of FIG. 6B includes a sloping point 616. As shown in FIG. 6B, the first surface 611 slopes away from a second surface 621 of the fluidic chamber 630 from the sloping point 616 towards an outlet 632 of the fluidic chamber 630.

As discussed above, removal of bubbles from the fluidic chamber 630 via a sloping surface is contingent on the orientation of the fluidic chamber 630 with respect to gravity. Specifically, removal of bubbles from the fluidic chamber 630 via the sloping surface is contingent on the sloping surface being located in the direction opposite the force of gravity with respect to the other surface of the fluidic chamber 630. Thus, the fluidic chamber 630 of FIG. 6B is oriented with respect to gravity such that the first surface 611 that includes the sloping point 616 is located in the direction opposite the force of gravity with respect to the second surface 621 of the fluidic chamber 630. In this orientation, bubbles formed within the fluidic chamber 630 are able to rise in the fluidic chamber 630 towards the first surface 611 and then travel along the first surface 611 of the fluidic chamber 630 towards the outlet 632 of the fluidic chamber 630 in a direction opposite the direction of the force of gravity, due to buoyant forces. In some embodiments, once the bubbles reach the outlet 632 of the fluidic chamber 630, the bubbles exit the fluidic chamber 630 via the outlet 632. Alternatively, the bubbles may remain within the fluidic chamber 630 along the first surface 611, but are displaced from the center of the volume of the fluidic chamber 630 such that they do not interfere, for example, with execution of an assay and/or with collection of assay results. An embodiment of the fluidic chamber 630 in which the second surface 621, rather than the first surface 611, of the fluidic chamber 630 includes a sloping point is discussed in detail below with regard to FIG. 6D.

Turning next to the embodiment of the fluidic chamber depicted in FIG. 6C, FIG. 6C depicts a third fluidic chamber 630, in accordance with an embodiment. The fluidic chamber 630 of FIG. 6C is similar to the fluidic chamber 630 of FIG. 6A. However, unlike the fluidic chamber 630 of FIG. 6A, instead of a first surface 611 of the fluidic chamber 630 of FIG. 6C having a sloping point, a second surface 621 of the fluidic chamber 630 of FIG. 6C includes a sloping point 616. As shown in FIG. 6C, the second surface 621 slopes away from the first surface 611 of the fluidic chamber 630 from the sloping point 616 towards an apex of the protrusion 614 of the fluidic chamber 630.

As discussed above, removal of bubbles from the fluidic chamber 630 via a sloping surface is contingent on the orientation of the fluidic chamber 630 with respect to gravity. Specifically, removal of bubbles from the fluidic chamber 630 via the sloping surface is contingent on the sloping surface being located in the direction opposite the force of gravity with respect to the other surface of the fluidic chamber 630. Thus, the fluidic chamber 630 of FIG. 6C is oriented with respect to gravity such that the second piece 620 that includes the sloping point 616 is located in the direction opposite the force of gravity with respect to the first surface 611 of the fluidic chamber 630. In this orientation, bubbles formed within the fluidic chamber 630 are able to rise in the fluidic chamber 630 towards the second surface 621 and then travel along the second surface 621 of the fluidic chamber 630 towards the apex of the protrusion 614 of the fluidic chamber 630 in a direction opposite the direction of the force of gravity, due to buoyant forces. When the bubbles reach the apex of the protrusion 614, the bubbles remain within the fluidic chamber 630 along the second surface 621, but are displaced from the center of the volume of the fluidic chamber 630 such that they do not interfere, for example, with execution of an assay and/or with collection of assay results.

Turning next to the embodiment of the fluidic chamber depicted in FIG. 6D, FIG. 6D depicts a fourth fluidic chamber 630, in accordance with an embodiment. The fluidic chamber 630 of FIG. 6D is similar to the fluidic chamber 630 of FIG. 6B. However, unlike the fluidic chamber 630 of FIG. 6B, instead of a first surface 611 of the fluidic chamber 630 of FIG. 6D having a sloping point, a second surface 621 of the fluidic chamber 630 of FIG. 6D includes a sloping point 616. As shown in FIG. 6D, the second surface 621 slopes away from the first surface 611 of the fluidic chamber 630 from the sloping point 616 towards an apex of the protrusion 614 of the fluidic chamber 630.

As discussed above, removal of bubbles from the fluidic chamber 630 via a sloping surface is contingent on the orientation of the fluidic chamber 630 with respect to gravity. Specifically, removal of bubbles from the fluidic chamber 630 via the sloping surface is contingent on the sloping surface being located in the direction opposite the force of gravity with respect to the other surface of the fluidic chamber 630. Thus, the fluidic chamber 630 of FIG. 6D is oriented with respect to gravity such that the second piece 620 that includes the sloping point 616 is located in the direction opposite the force of gravity with respect to the first surface 611 of the fluidic chamber 630. In this orientation, bubbles formed within the fluidic chamber 630 are able to rise in the fluidic chamber 630 towards the second surface 621 and then travel along the second surface 621 of the fluidic chamber 630 towards the apex of the protrusion 614 of the fluidic chamber 630 in a direction opposite the direction of the force of gravity, due to buoyant forces. When the bubbles reach the apex of the protrusion 614, the bubbles remain within the fluidic chamber 630 along the second surface 621, but are displaced from the center of the volume of the fluidic chamber 630 such that they do not interfere, for example, with execution of an assay and/or with collection of assay results.

Turning next to the embodiment of the fluidic chamber depicted in FIG. 6E, FIG. 6E depicts a fifth fluidic chamber 630, in accordance with an embodiment. The fluidic chamber 630 of FIG. 6E is similar to the fluidic chamber 530 of FIG. 5E. However, unlike the fluidic chamber 530 of FIG. 5E, a first surface 611 of the fluidic chamber 630 of FIG. 6E includes a sloping point 616. As shown in FIG. 6E, the first surface 611 slopes away from a second surface 621 of the fluidic chamber 630 from the sloping point 616 towards an apex of the second protrusion 624 of the fluidic chamber 630.

As discussed above, removal of bubbles from the fluidic chamber 630 via a sloping surface is contingent on the orientation of the fluidic chamber 630 with respect to gravity. Specifically, removal of bubbles from the fluidic chamber 630 via the sloping surface is contingent on the sloping surface being located in the direction opposite the force of gravity with respect to the other surface of the fluidic chamber 630. Thus, the fluidic chamber 630 of FIG. 6E is oriented with respect to gravity such that the first surface 611 that includes the sloping point 616 is located in the direction opposite the force of gravity with respect to the second surface 621 of the fluidic chamber 630. In this orientation, bubbles formed within the fluidic chamber 630 are able to rise in the fluidic chamber 630 towards the first surface 611 and then travel along the first surface 611 of the fluidic chamber 630 towards the apex of the second protrusion 624 of the fluidic chamber 630 in a direction opposite the direction of the force of gravity, due to buoyant forces. When the bubbles reach the apex of the second protrusion 624, the bubbles remain within the fluidic chamber 630 along the first surface 611, but are displaced from the center of the volume of the fluidic chamber 630 such that they do not interfere, for example, with execution of an assay and/or with collection of assay results. An embodiment of the fluidic chamber 630 in which the second surface 621, rather than the first surface 611, of the fluidic chamber 630 includes a sloping point is discussed in detail below with regard to FIG. 6F.

Turning finally to the embodiment of the fluidic chamber depicted in FIG. 6F, FIG. 6F depicts a sixth fluidic chamber 630, in accordance with an embodiment. The fluidic chamber 630 of FIG. 6F is similar to the fluidic chamber 630 of FIG. 6E. However, unlike the fluidic chamber 630 of FIG. 6E, instead of a first surface 611 of the fluidic chamber 630 of FIG. 6F having a sloping point, a second surface 621 of the fluidic chamber 630 of FIG. 6F includes a sloping point 616. As shown in FIG. 6F, the second surface 621 slopes away from the first surface 611 of the fluidic chamber 630 from the sloping point 616 towards an apex of the protrusion 614 of the fluidic chamber 630.

As discussed above, removal of bubbles from the fluidic chamber 630 via a sloping surface is contingent on the orientation of the fluidic chamber 630 with respect to gravity. Specifically, removal of bubbles from the fluidic chamber 630 via the sloping surface is contingent on the sloping surface being located in the direction opposite the force of gravity with respect to the other surface of the fluidic chamber 630. Thus, the fluidic chamber 630 of FIG. 6F is oriented with respect to gravity such that the second piece 620 that includes the sloping point 616 is located in the direction opposite the force of gravity with respect to the first surface 611 of the fluidic chamber 630. In this orientation, bubbles formed within the fluidic chamber 630 are able to rise in the fluidic chamber 630 towards the second surface 621 and then travel along the second surface 621 of the fluidic chamber 630 towards the apex of the protrusion 614 of the fluidic chamber 630 in a direction opposite the direction of the force of gravity, due to buoyant forces. When the bubbles reach the apex of the protrusion 614, the bubbles remain within the fluidic chamber 630 along the second surface 621, but are displaced from the center of the volume of the fluidic chamber 630 such that they do not interfere, for example, with execution of an assay and/or with collection of assay results.

Note that while the embodiments of the fluidic chamber 630 shown in FIGS. 6A-F include only a single sloping point, in alternative embodiments, both surfaces of a fluidic chamber (e.g., a first surface and a second surface) may include a sloping point. In such embodiments in which both surfaces of a fluidic chamber include a sloping point, to remove and/or displace bubbles from the fluidic chamber, the fluidic chamber may be oriented such that either the first surface or the second surface is located in the direction opposite the force of gravity with respect to the other surface of the fluidic chamber.

Furthermore, prior to and following bubble removal from a fluidic chamber, the fluidic chamber may be oriented in any orientation. In other words, the fluidic chamber may be oriented as described above only during bubble removal, and may be oriented alternatively at other time points. Orientation of a fluidic chamber may be performed manually, mechanically, or by any other means.

FIG. 7A depicts a fluidic chamber 730 configured to avoid bubble formation during filling of the fluidic chamber 730 with a liquid, in accordance with an embodiment. The fluidic chamber 730 is formed by the operative coupling of a first piece 710 and a second piece 720. In the embodiment shown in FIG. 7A, the first piece 710 and the second piece 720 are operatively coupled by a gasket 734. A first surface 711 of the first piece 710 and a second surface 721 of the second piece 720 bound a volume of the fluidic chamber 730. The fluidic chamber 730 includes an inlet 731 and an outlet 732.

The first piece 710 includes a protrusion 713 that is bounded by the first surface 711 of the first piece 710. The protrusion 713 protrudes into the fluidic chamber 730 such that there is a distance of minimal approach between an apex of the protrusion 714 and the second surface 721 of the second piece 720. In the embodiment depicted in FIG. 7A, the distance of minimal approach between the apex of the protrusion 714 and the second surface 721 of the second piece 720 is less than a largest dimension of the cross-sectional area of the volume of the fluidic chamber 730 at a transverse plane of the fluidic chamber 730.

The protrusion 713 forms a channel 715 that extends from the inlet 731 of the fluidic chamber 730 to the apex of the protrusion 714. Both the inlet 731 and the outlet 732 of the fluidic chamber 730 are formed in the first piece 710 of the fluidic chamber 730 such that the apex of the protrusion 714 is located diagonally across the volume of the fluidic chamber 730 from the outlet 732, and such that a maximum distance of travel through the volume of the fluidic chamber 730 exists between the inlet 731 and the outlet 732.

A cross-sectional area of the volume of the fluidic chamber 730 increases from the apex of the protrusion 714, where the cross-sectional area is defined in part by the distance of minimal approach, to the transverse plane of the fluidic chamber 730, and decreases from the transverse plane of the fluidic chamber 730 to the outlet 732 of the fluidic chamber 730.

The first surface 711 of the fluidic chamber 730 includes a sloping point 716. As shown in FIG. 7A, the first surface 711 slopes away from the second surface 721 of the fluidic chamber 730 from the sloping point 716 towards the outlet 732 of the fluidic chamber 730.

As shown in FIG. 7A, the corners of the fluidic chamber 730 are radiused. As a result, the first surface 711 of the first piece 710 has one or more primary radii of curvature. For example, the first surface 711 of the first piece 710 includes a primary radius of curvature 712. Similarly, the second surface 721 of the second piece 720 has one or more secondary radii of curvature. For example, the second surface 721 of the second piece 720 includes a secondary radius of curvature 721. As discussed in further detail below with regard to FIG. 7B, each of the primary radii of curvature and the secondary radii of curvature, including the primary radius of curvature 712 and the secondary radius of curvature 722, are greater than a radius of curvature of the meniscus of a liquid filling the fluidic chamber 730.

FIG. 7B depicts the fluidic chamber 730 of FIG. 7A, during filling of the fluidic chamber 730 with a liquid 750, in accordance with an embodiment. Specifically, FIG. 7B depicts expansion of a meniscus of the liquid 750 over time, as the liquid 750 fills the fluidic chamber 730. The expansion of a meniscus of the liquid 750 over time is depicted as concentric arcs. The smallest concentric arc that begins at the apex of the protrusion 714 is the meniscus of the liquid 750 at a first time point. The mid-sized concentric arc is the meniscus of the liquid 750 at a second time point subsequent to the first time point. The largest concentric arc is the meniscus of the liquid 750 at a third time point subsequent to the second time point.

As shown in FIG. 7B, because the cross-sectional area of the volume of the fluidic chamber 730 increases from the apex of the protrusion 714 to the transverse plane and decreases from the transverse plane to the outlet 732, the liquid 750 gradually fills the fluidic chamber 730 while avoiding bubble formation within the liquid 750. Specifically, because the cross-sectional area of the volume of the fluidic chamber 730 increases from the apex of the protrusion 714 to the transverse plane and decreases from the transverse plane to the outlet 732, the liquid 750 gradually fills the volume of the fluidic chamber 730 such that a radius of curvature of the meniscus of the liquid 751 increases from the apex of the protrusion 714 to the transverse plane of the fluidic chamber 730, but does not surpass a radius of curvature of the first and second surfaces 711 and 721 of the fluidic chamber 730. For example, as shown in FIG. 7B, at each of the three time points, the radius of curvature of the meniscus of the liquid 751 is smaller than the secondary radius of curvature 722 of the fluidic chamber 730. This minimization of the radius of curvature of the liquid 751 filling the fluidic chamber 730 relative to the radii of curvature of the surfaces of the fluidic chamber 730, as enabled by the shape of the fluidic chamber 730, minimizes the trapping of bubbles within the fluidic chamber 730 during filling.

FIG. 8A depicts a fluidic chamber 830 with a transverse plane 833, in accordance with an embodiment. As discussed throughout this disclosure, a transverse plane of a fluidic chamber is a plane of the fluidic chamber at which a cross-sectional area of the volume of the fluidic chamber transitions between increasing and decreasing in magnitude. More specifically, as shown in FIG. 8A, the transverse plane 833 of the fluidic chamber 830 is a plane of the fluidic chamber 830 at which a cross-sectional area A of the volume of the fluidic chamber 830 transitions between increasing and decreasing in magnitude, along a length 1 of the fluidic chamber. This functional definition of the transverse plane 833 is further exemplified in FIG. 8B.

FIG. 8B is a line graph that depicts the relationship between the cross-sectional area A of the volume of the fluidic chamber 830 and the length 1 along the fluidic chamber 830, in accordance with an embodiment. As shown in FIG. 8B, the cross-sectional area A of the volume of the fluidic chamber 830 increases along the length 1 of the fluidic chamber 830 until the transverse plane 833 is reached. Once the transverse plane 833 is reached, the cross-sectional area A of the volume of the fluidic chamber 830 decreases along the length 1 of the fluidic chamber 830.

As a result of this functional definition of the transverse plane 833, the cross-sectional area A of the volume of the fluidic chamber 830 is at a maximum magnitude at the transverse plane 833. And therefore, as a liquid fills the fluidic chamber 830, a radius of curvature of a meniscus of the liquid filling the fluidic chamber 830 reaches a maximum magnitude at the transverse plane 833 of the volume of the fluidic chamber 830.

Note that despite the functional definition of the transverse plane as the plane of a fluidic chamber at which a cross-sectional area of the volume of the fluidic chamber transitions between increasing and decreasing in magnitude, in some embodiments, the cross-sectional area of the volume of the fluidic chamber may not strictly transition between increasing and decreasing in magnitude. Specifically, in some embodiments, up to x % of a total cross-sectional volume of a fluidic chamber can defy the increasing-decreasing pattern. For example, a cross-sectional area of a volume of a fluidic chamber may increase in magnitude, become constant in magnitude for up to x % of a total cross-sectional volume of the fluidic chamber, and then decrease in magnitude. These alternative embodiments in which a cross-sectional area of the volume of a fluidic chamber does not strictly transition between increasing and decreasing in magnitude are still operable to avoid formation of bubbles during filling of the fluidic chamber with a liquid as described herein.

### Examples

FIG. 9 depicts an exemplar fluidic chamber 930 at a plurality of sequential time points during filling of the fluidic chamber 930 with a liquid 950, in accordance with an embodiment. Specifically, FIG. 9 depicts the fluidic chamber 930 at time points t=0 seconds, 0.2 seconds, 0.3 seconds, 0.5 seconds, 0.8 seconds, 0.9 seconds, 1.1 seconds, and 1.3 seconds during filling of the fluidic chamber 930 with the liquid 950. The liquid 950 in FIG. 9 is shown as a dark fluid.

As shown in FIG. 9, a first piece 910 is operatively coupled to a second piece 920 to form the fluidic chamber 930. A volume of the fluidic chamber 930 is bounded by a first surface 911 of the first piece 910 and a second surface 921 of the second piece 920. The fluidic chamber 930 includes an inlet 931 and an outlet 932. The first piece 910 includes a protrusion 913 bounded by the first surface 911. The protrusion 913 protrudes into the fluidic chamber 930 such that there is a distance of minimal approach between an apex of the protrusion 914 and the second surface 921 of the second piece 920. The protrusion 913 also forms a channel 915 that extends from the inlet 931 to the apex of the protrusion 914.

A maximum possible distance of travel through the volume of the fluidic chamber 930 exists between the inlet 931 and the outlet 932. Additionally, a cross-sectional area of the volume of the fluidic chamber 930 increases from the apex of the protrusion 914 to a transverse plane of the fluidic chamber 930, and decreases from the transverse plane to the outlet 932.

At time point t=0 seconds, the liquid 950 has not been introduced into the inlet 931 of the fluidic chamber 930, and thus has not yet entered the fluidic chamber 930.

At time point t=0.2 seconds, the liquid 950 has been introduced into the inlet 931 of the fluidic chamber 930, and has flowed from the inlet 931 and into the channel 915 in a direction of the apex of the protrusion 914.

At time point t=0.3 seconds, the liquid 950 has flowed through the channel 915 and has reached the apex of the protrusion 914.

At time point t=0.5 seconds, the liquid 950 begins to gradually fill the volume of the fluidic chamber 930. Specifically, the liquid 950 flows through the cross-sectional area of the volume of the fluidic chamber 930 that is defined in part by the distance of minimal approach between the apex of the protrusion 914 and the second surface 921 of the second piece 920, and gradually fills the volume of the fluidic chamber 930 such that a radius of curvature of a meniscus of the liquid 951 increases from the apex of the protrusion 914, where the radius of curvature of the meniscus of the liquid 951 is constrained by the distance of minimal approach, to a transverse plane of the fluidic chamber 930, where the cross-sectional area of the volume of the fluidic chamber 930 is at a maximum. At the time point t=0.5 seconds, the liquid 950 has not yet reached the transverse plane of the fluidic chamber 930, and thus the radius of curvature of the meniscus of the liquid 951 is not yet at a maximum. In other words, at the time point t=0.5 seconds the radius of curvature of the meniscus of the liquid 951 is still increasing in magnitude.

At the time point t=0.8 seconds, the liquid 950 has reached the transverse plane of the fluidic chamber 930, and thus the radius of curvature of the meniscus of the liquid 951 is at a maximum magnitude. Note however, that the radius of curvature of the meniscus of the liquid 951 does not surpass a radius of curvature of the first surface 911 or the second surface 921—even at its maximum magnitude. As a result of this discrepancy between the radius of curvature of the meniscus of the liquid 951 and the radii of curvature of the first surface 911 or the second surface 921, no bubbles are trapped within the fluidic chamber 930.

At the time point t=0.9 seconds, the liquid 950 has flowed past the transverse plane of the fluidic chamber 930, and continues to gradually fill the volume of the fluidic chamber 930. However, the radius of curvature of the meniscus of the liquid 951 decreases as the liquid 950 travels from the transverse plane, where the radius of curvature of the meniscus of the liquid 951 was at a maximum magnitude due to the maximum cross-sectional area of the volume of the fluidic chamber 930 at the transverse plane, to the outlet 932 of the fluidic chamber 930. Thus, at the time point t=0.9 seconds, the radius of curvature of the meniscus of the liquid 951 is decreasing in magnitude.

At the time point t=1.1 seconds, the liquid 950 continues to gradually fill the volume of the fluidic chamber 930. As the liquid 950 moves from the transverse plane of the fluidic chamber 930 towards the outlet 932 of the fluidic chamber 930, the radius of curvature of the meniscus of the liquid 951 continues to decrease.

At the time point t=1.3 seconds, the liquid 950 has reached the outlet 932 of the fluidic chamber 930. In some embodiments, such as the embodiment depicted in FIG. 9, the liquid 950 reaches the outlet 932 of the fluidic chamber 930 when the volume of the fluidic chamber 930 is substantially filled with the liquid 950. As used herein, the term “substantially filled” means at least 90% filled. In alternative embodiments, the liquid 950 may reach the outlet 932 of the fluidic chamber 930 before the fluidic chamber 930 is substantially filled.

As shown in FIG. 9, in some embodiments, the liquid 950 may exit the fluidic chamber 930 via the outlet 932 when the liquid 950 reaches the outlet 932. In further embodiments in which a plurality of fluidic chambers are in fluidic communication with one another via at least one of an inlet and an outlet of each fluidic chamber as described above with regard to FIG. 1, when liquid exits a first fluidic chamber via an outlet of the first fluidic chamber, the liquid may travel into a second fluidic chamber via an inlet of the second fluidic chamber that is in fluidic communication with the outlet of the first fluidic chamber. In alternative embodiments, the liquid 950 may not be able to exit the fluidic chamber 930.

The fluidic chamber 930 of FIG. 9 is configured similarly to the fluidic chamber 530 in FIGS. 5B and 5D. However, as discussed above with regard to FIGS. 5A-F, a fluidic chamber may be configured differently than the fluidic chamber 930 of FIG. 9. Specifically, as discussed above with regard to FIGS. 5A-F, a fluidic chamber may have one or more protrusions and channels, and these protrusion(s) and channel(s) may be alternatively positioned. Filling of a fluidic chamber with a liquid may vary slightly based on the specific configuration of the fluidic chamber, as discussed in further detail below.

For instance, turning back to the embodiment of the fluidic chamber 530 in FIGS. 5A and 5C, a liquid may be introduced into the inlet 531 of the fluidic chamber 530, upon the liquid gradually fills the volume of the fluidic chamber 530 such that a radius of curvature a meniscus of the liquid increases from the inlet 531 of the fluidic chamber 530 to the transverse plane of the fluidic chamber 530, and decreases from the transverse plane of the fluidic chamber 530 to the apex of the protrusion 514, but does not surpass a radius of curvature of one or more surfaces of the fluidic chamber 530, thereby minimizing the trapping of bubbles within the fluidic chamber 530 during filling. In such embodiments, upon reaching the apex of the protrusion 514, the liquid may flow into the channel 515 formed by the protrusion 513, and towards the outlet 532 of the fluidic chamber 530. And in some further embodiments, upon reaching the outlet 532 of the fluidic chamber 530, the liquid exits the fluidic chamber 530 via the outlet 532.

Turning next to the embodiment of the fluidic chamber 530 in FIGS. 5E and 5F, a liquid may be introduced into the inlet 531 of the fluidic chamber 530, upon the liquid flows from the inlet 531 of the fluidic chamber 530 to the apex of the protrusion 514 (or the apex of the second protrusion 524) via the channel 515 (or the second channel 525). Then, upon reaching the apex of the protrusion 514 (or the second apex of the protrusion 524), the liquid gradually fills the volume of the fluidic chamber 530 such that a radius of curvature a meniscus of the liquid increases from the apex of the protrusion 514 (or the apex of the second protrusion 524) to the transverse plane of the fluidic chamber 530, and decreases from the transverse plane of the fluidic chamber 530 to the apex of the second protrusion 524 (or the apex of the protrusion 514), but does not surpass a radius of curvature of one or more surfaces of the fluidic chamber 530, thereby minimizing the trapping of bubbles within the fluidic chamber 530 during filling. In such embodiments, upon reaching the apex of the second protrusion 524 (or the apex of the protrusion 514), the liquid may flow into the second channel 525 (or the channel 515) formed by the second protrusion 523 (or the protrusion 513), and towards the outlet 532 of the fluidic chamber 530. And in some further embodiments, upon reaching the outlet 532 of the fluidic chamber 530, the liquid exits the fluidic chamber 530 via the outlet 532.

### Optical Interrogation of Fluidic Chambers

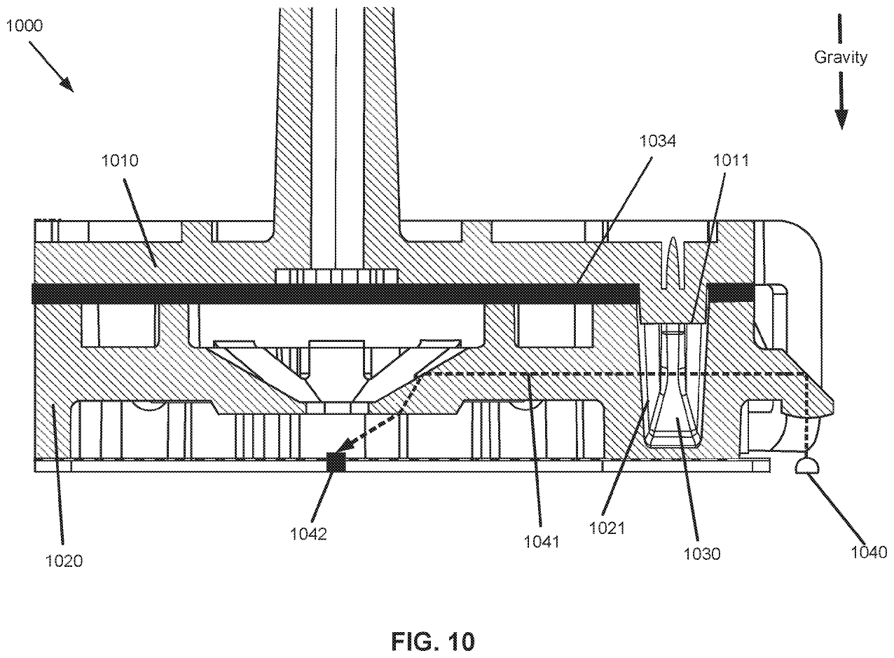

FIG. 10 is a cross-section of an assembly 1000 for avoiding bubble formation in a fluidic chamber 1030 of the assembly 1000, during filling of the fluidic chamber 1030 with a liquid, and for interrogation of the liquid contained within the fluidic chamber 1030, in accordance with an embodiment. The assembly 1000 includes a first piece 1010 and a second piece 1020 that are operatively coupled to one another by a gasket 1034 to form the fluidic chamber 1030. A first surface 1011 of the first piece 1010 and a second surface 1021 of the second piece 1020 bound a volume of the fluidic chamber 1030. The fluidic chamber 1030 may be configured and filled with a liquid according to one or more of the embodiments described above.

Interrogation of the liquid contained within the fluidic chamber 1030 is performed at least in part by a light emitting element 1040. The light emitting element 1040 is configured to interrogate the liquid contained within the fluidic chamber 1030 by transmitting a light in a direction of the fluidic chamber 1030 via an interrogation pathway 1041 that is orthogonal to the force of gravity. In other words, interrogation of the fluidic chamber 1030 occurs through a side of the fluidic chamber 1030, rather than at a surface of the fluidic chamber 1030. This enables analysis of the bulk volume of the fluidic chamber 1030, thereby yielding more accurate and reliable results.

As discussed in detail throughout this disclosure, the accuracy of interrogation of a liquid can be compromised by the presence of bubbles in the liquid. To mitigate this problem, the fluidic chamber 1030 is configured not only to prevent formation of bubbles, but in some embodiments to remove and/or displace bubbles that do form in the liquid contained within the fluidic chamber 1030. Specifically, as discussed above with regard to FIGS. 6A-F, in some embodiments, to remove and/or displace bubbles from liquid contained within the fluidic chamber 1030, a surface of the fluidic chamber 1030 contains a sloping point, and the fluidic chamber 1030 is oriented such that the surface containing the sloping point is located in a direction that is opposite the direction of the force of gravity relative to the other surface of the fluidic chamber 1030. With this configuration and orientation of the fluidic chamber 1030, due to buoyant forces, bubbles that form in the liquid contained within the fluidic chamber 1030 can rise in the fluidic chamber 1030 towards the surface containing the sloping point, and then travel along the sloping surface in a direction opposite the direction of the force of gravity and towards one of an inlet, an outlet, or an apex of a protrusion of the fluidic chamber 1030, where the bubbles can escape the fluidic chamber 1030, or at least be displaced from the center of the volume of the fluidic chamber 1030.

In such embodiments in which the fluidic chamber 1030 is configured and oriented to remove and/or displace bubbles from the liquid contained within the fluidic chamber 1030 as described above, the positioning of the interrogation pathway 1041 as orthogonal to the force of gravity, and thus to the path of buoyancy for bubbles, enables interrogation of the liquid contained within the fluidic chamber 1030 without interference of bubbles. Specifically, in embodiments in which the fluidic chamber 1030 is configured and oriented to remove and/or displace bubbles from the liquid contained within the fluidic chamber 1030 as described above, bubbles either escape the fluidic chamber 1030 or are at least removed from the interrogation pathway 1041 of the fluidic chamber 1030 via the path of buoyancy. By interrogating the liquid contained within the fluidic chamber 1030 via the interrogation pathway 1041 that is orthogonal to the force of gravity and thus to the path of buoyancy of the bubbles, the interrogation pathway 1041 avoids bubbles in the liquid of the fluidic chamber 1030. As a result, bubbles do not interfere with the interrogation of the liquid, thereby improving the accuracy of the interrogation.

In some embodiments, at least a portion of one of the first surface 1011 and the second surface 1021 comprises a transparent material, and the interrogation pathway 1041 extends through the transparent material such that the light emitted by the light emitting element 1040 along the interrogation pathway 1041 passes through the transparent material. In some further embodiments, one or more of a light guide, a light filter, and a lens may be located along the interrogation pathway 1041 between the light emitting element 1040 and the fluidic chamber 1030, and may be used to modify the light transmitted towards the fluidic chamber 1030 via the interrogation pathway 1041.

After the light passes through the fluidic chamber 1030 along the interrogation pathway 1041, the light may be used to detect optical properties and/or changes in optical properties of the liquid contained in the fluidic chamber 1030. As used herein, optical properties refer to one or more optically-recognizable characteristics, such as a characteristic resulting from wavelength and/or frequency of radiation, e.g., light, emitted by or transmitted through a sample, prior to, during, or following an assay reaction carried on using said sample, such as color, absorbance, reflectance, scattering, fluorescence, phosphorescence, etc. These detected optical properties may be used to characterize the liquid contained within the fluidic chamber 1030 and/or to characterize an assay involving the liquid contained within the fluidic chamber 1030.

In certain embodiments, such as the embodiment depicted in FIG. 10, a photosensor 1042 may be positioned along the interrogation pathway 1041 to receive the light after it passes through the fluidic chamber 1030 and to subsequently detect one or more optical properties of the liquid contained within the fluidic chamber 1030. In alternative embodiments, the assembly 1000 may not comprise the photosensor 1042. In alternative embodiments, the light that passes through the fluidic chamber 1030 may be received directly by an eye of a user such that the user may detect one or more optical properties of the liquid contained within the fluidic chamber 1030, and use those detected optical properties to characterize the liquid contained within the fluidic chamber 1030.

### CONCLUSION

Upon reading this disclosure, those of skill in the art will appreciate still additional alternative structural and functional designs through the disclosed principles herein. Thus, while particular embodiments and applications have been illustrated and described, it is to be understood that the disclosed embodiments are not limited to the precise construction and components disclosed herein. Various modifications, changes and variations, which will be apparent to those skilled in the art, can be made in the arrangement, operation and details of the method and assembly disclosed herein without departing from the spirit and scope defined in the appended claims.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Some embodiments can be described using the expression “coupled” and “connected” along with their derivatives. For example, some embodiments can be described using the term “coupled” to indicate that two or more elements are in direct physical or electrical contact. The term “coupled,” however, can also mean that two or more elements are not in direct contact with each other, but yet still co-operate or interact with each other. The embodiments are not limited in this context unless otherwise explicitly stated.

As used herein, the terms “comprises,” “comprising,” “includes,” “including,” “has,” “having” or any other variation thereof, are intended to cover a non-exclusive inclusion. For example, a process, method, article, or assembly that comprises a list of elements is not necessarily limited to only those elements but can include other elements not expressly listed or inherent to such process, method, article, or assembly. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the invention. This description should be read to include one or at least one and the singular also includes the plural unless it is obvious that it is meant otherwise.

Some portions of this description describe the embodiments of the invention in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules can be embodied in software, firmware, hardware, or any combinations thereof.

Any of the steps, operations, or processes described herein can be performed or implemented with one or more hardware or software modules, alone or in combination with other devices. In one embodiment, a software module is implemented with a computer program product including a computer-readable non-transitory medium containing computer program code, which can be executed by a computer processor for performing any or all of the steps, operations, or processes described.

Embodiments of the invention can also relate to a product that is produced by a computing process described herein. Such a product can include information resulting from a computing process, where the information is stored on a non-transitory, tangible computer readable storage medium and can include any embodiment of a computer program product or other data combination described herein.

### FIGURES REFERENCE NUMBER LIST

| Item | Last 2 Digits |
|---|---|
| assembly | 00 |
| first piece | 10 |
| first surface | 11 |
| primary radius of curvature | 12 |
| protrusion | 13 |
| apex of the protrusion | 14 |
| channel | 15 |
| sloping point | 16 |
| second piece | 20 |
| second surface | 21 |
| secondary radius of curvature | 22 |
| second protrusion | 23 |
| apex of the second protrusion | 24 |
| second channel | 25 |
| fluidic chamber | 30 |
| inlet | 31 |
| outlet | 32 |
| transverse plane | 33 |
| gasket | 34 |
| lyophilized reagents | 35 |
| light emitting element | 40 |
| interrogation pathway | 41 |
| photosensor | 42 |
| liquid | 50 |
| radius of curvature of liquid meniscus | 51 |

1. An assembly configured to avoid bubble formation in a fluidic chamber of the assembly during filling of the fluidic chamber with a liquid, the assembly comprising:
a first piece comprising:
a first surface; and
a protrusion, the first surface of the first piece bounding the protrusion; and
a second piece comprising a second surface,
wherein the first piece and the second piece are operatively coupled to one another to form the fluidic chamber of the assembly, the fluidic chamber comprising:
an inlet;
an outlet;
a volume bounded by the first and second surfaces,
wherein the protrusion of the first piece protrudes into the volume of the fluidic chamber such that there is a distance of minimal approach between an apex of the protrusion and the second surface of the second piece; and
a channel formed by the protrusion of the first piece, the channel extending from one of the inlet and the outlet to the apex of the protrusion,
wherein the inlet and the outlet of the fluidic chamber are positioned in the fluidic chamber such that a maximum distance of travel through the volume of the fluidic chamber exists between the inlet and the outlet, and
wherein a cross-sectional area of the volume of the fluidic chamber increases from the apex of the protrusion to a transverse plane of the fluidic chamber and decreases from the transverse plane of the fluidic chamber to the other one of the inlet and the outlet of the fluidic chamber.

2. The assembly of claim 1, wherein the distance of minimal approach between the apex of the protrusion and the second surface of the second piece is less than a largest dimension of the cross-sectional area of the volume of the fluidic chamber at the transverse plane of the fluidic chamber.

3. The assembly of any one of claims 1-2, wherein the one of the inlet and the outlet of the fluidic chamber comprises the inlet, and the other of the one of the inlet and the outlet of the fluidic chamber comprises the outlet.

4. The assembly of any one of claims 1-2, wherein the one of the inlet and the outlet of the fluidic chamber comprises the outlet, and the other of the one of the inlet and the outlet of the fluidic chamber comprises the inlet.

5. The assembly of any one of claims 1-4, wherein the apex of the protrusion is located diagonally across the volume of the fluidic chamber from the other of the one of the inlet and the outlet.

6. The assembly of any one of claims 1-5, wherein the inlet and the outlet of the fluidic chamber are formed in the first piece of the assembly.

7. The assembly of any one of claims 1-6, wherein the assembly is oriented such that the second piece is located in the direction of the force of gravity with respect to the first piece.

8. The assembly of claim 7, the assembly further configured to remove bubbles from the fluidic chamber, wherein the first surface of the first piece slopes away from the second surface of the second piece at a non-zero slope towards the other one of the inlet and the outlet of the fluidic chamber.

9. The assembly of any one of claims 1-6, wherein the assembly is oriented such that the first piece is located in the direction of the force of gravity with respect to the second piece.

10. The assembly of claim 9, the assembly further configured to remove bubbles from the fluidic chamber, wherein the second surface of the second piece slopes away from the first surface of the first piece at a non-zero slope towards the apex of the protrusion of the first piece.

11. An assembly configured to avoid bubble formation in a fluidic chamber of the assembly during filling of the fluidic chamber with a liquid, the assembly comprising:
a first piece comprising:
a first surface; and
a protrusion, the first surface of the first piece bounding the protrusion; and
a second piece comprising:
a second surface; and
a second protrusion, the second surface of the second piece bounding the second protrusion,
wherein the first piece and the second piece are operatively coupled to one another to form the fluidic chamber of the assembly, the fluidic chamber comprising:
an inlet;
an outlet;
a volume bounded by the first and second surfaces,
wherein the protrusion of the first piece protrudes into the volume of the fluidic chamber such that there is a distance of minimal approach between an apex of the protrusion and the second surface of the second piece,
wherein the second protrusion of the second piece protrudes into the volume of the fluidic chamber such that there is a second distance of minimal approach between an apex of the second protrusion and the first surface of the first piece;
a channel formed by the protrusion of the first piece, the channel extending from one of the inlet and the outlet to the apex of the protrusion; and
a second channel formed by the second protrusion of the second piece, the second channel extending from the other one of the inlet and the outlet of the fluidic chamber to the apex of the second protrusion,
wherein the inlet and the outlet of the fluidic chamber are positioned in the fluidic chamber such that a maximum distance of travel through the volume of the fluidic chamber exists between the inlet and the outlet, and
wherein a cross-sectional area of the volume of the fluidic chamber increases from the apex of the protrusion to a transverse plane of the fluidic chamber and decreases from the transverse plane to the apex of the second protrusion.

12. The assembly of claim 11, wherein the one of the inlet and the outlet of the fluidic chamber comprises the inlet, and the other of the one of the inlet and the outlet of the fluidic chamber comprises the outlet.

13. The assembly of claim 11, wherein the one of the inlet and the outlet of the fluidic chamber comprises the outlet, and the other of the one of the inlet and the outlet of the fluidic chamber comprises the inlet.

14. The assembly of any one of claims 11-13, wherein the distance of minimal approach between the apex of the protrusion and the second surface of the second piece is less than a largest dimension of the cross-sectional area of the volume of the fluidic chamber at the transverse plane of the fluidic chamber.

15. The assembly of any one of claims 11-14, wherein the apex of the second protrusion is located diagonally across the volume of the fluidic chamber from the apex of the protrusion.

16. The assembly of any one of claims 11-15, wherein the second distance of minimal approach between the apex of the second protrusion and the first surface of the first piece is less than the largest dimension of the cross-sectional area of the volume of the fluidic chamber at the transverse plane of the fluidic chamber.

17. The assembly of any one of claims 11-16, wherein the inlet of the fluidic chamber is formed in the first piece of the assembly and the outlet of the fluidic chamber is formed in the second piece of the assembly.

18. The assembly of any one of claims 11-17, wherein the assembly is oriented such that the second piece is located in the direction of the force of gravity with respect to the first piece.

19. The assembly of claim 18, the assembly further configured to remove bubbles from the fluidic chamber, wherein the first surface of the first piece slopes away from the second surface of the second piece at a non-zero slope towards the apex of the second protrusion of the second piece.

20. The assembly of any one of claims 11-17, wherein the assembly is oriented such that the first piece is located in the direction of the force of gravity with respect to the second piece.

21. The assembly of claim 20, the assembly further configured to remove bubbles from the fluidic chamber, wherein the second surface of the second piece slopes away from the first surface of the first piece at a non-zero slope towards the apex of the protrusion of the first piece.

22. The assembly of any one of claims 1-21, wherein a shape of the volume of the fluidic chamber substantially comprises a quadrilateral prism.

23. The assembly of claim 22, wherein one or more corners of the quadrilateral prism are radiused.

24. The assembly of any one of claims 1-23, wherein the first surface of the first piece has one or more primary radii of curvature and the second surface of the second piece has one or more secondary radii of curvature, each of the primary radii of curvature and secondary radii of curvature being greater than a radius of curvature of a meniscus of the liquid filling the fluidic chamber.

25. The assembly of any one of claims 1-24, wherein the first surface of the first piece and the second surface of the second piece have a roughness value of less than 25 micro-inches.

26. The assembly of any one of claims 1-25, wherein at least one of the first piece and the second piece is injection molded.

27. The assembly of any one of claims 1-26, wherein at least one of the first piece and the second piece is formed by one of replica casting, vacuum-forming, machining, chemical etching, and physical etching.

28. The assembly of any one of claims 1-27, wherein at least one of the first piece and the second piece comprises one of plastic, metal, and glass.

29. The assembly of any one of claims 1-28, wherein at least one of the first piece and the second piece comprises one of a hydrophobic and an oleophobic material.

30. The assembly of any one of claims 1-29, wherein the contact angle between the liquid filling the fluidic chamber and at least one of the first surface and the second surface of the fluidic chamber is greater than 90 degrees.

31. The assembly of any one of claims 1-30, further comprising a gasket located between the first piece and the second piece, the gasket operatively coupled to the first piece and the second piece to form fluid seals in the fluidic chamber.

32. The assembly of claim 31, wherein the gasket comprises thermoplastic elastomeric (TPE) overmolding.

33. The assembly of any one of claims 31-32, wherein a volume of the gasket is compressed by 5%-25% when the first piece and the second piece are operatively coupled.

34. The assembly of any one of claims 1-33, wherein the first piece and the second piece are operatively coupled by one or more of compression, ultrasonic welding, thermal welding, laser welding, solvent bonding, adhesives, and heat staking.

35. The assembly of any one of claims 1-34, wherein the volume of the fluidic chamber is between 1 uL and 1000 uL.

36. The assembly of claim 35, wherein the volume of the fluidic chamber is on the order of 30 uL.

37. The assembly of any one of claims 1-36, wherein the fluidic chamber contains dried or lyophilized reagents.

38. The assembly of claim 37, wherein the dried or lyophilized reagents comprise assay reagents.

39. The assembly of claim 38, wherein the assay reagents comprise a nucleic acid amplification enzyme and a DNA primer.

40. The assembly of any one of claims 1-39, wherein the assembly further comprises:
a light emitting element configured to interrogate the liquid contained in the fluidic chamber using light that travels via an interrogation pathway that is orthogonal to the force of gravity.

41. The assembly of claim 40, wherein at least a portion of one of the first and second surfaces comprises a transparent material, and wherein the interrogation pathway via which the light emitting element is configured to interrogate the liquid contained in the fluidic chamber extends through the transparent material.

42. The assembly of claim 41, wherein the one of the first and second surfaces comprises the second surface.

43. The assembly of any one of claims 41-42, further comprising one or more of a light guide, a light filter, and a lens located along the interrogation pathway between the light emitting element and the fluidic chamber.

44. The assembly of any one of claims 1-43, wherein the operative coupling of the first and the second pieces forms a plurality of fluidic chambers.

45. The assembly of claim 44, wherein each of the plurality of fluidic chambers is in fluidic communication with at least one other fluidic chamber of the plurality of fluidic chambers via at a fluidic connection between one of an inlet and an outlet of the fluidic chamber, and the other of the one of the inlet and the outlet of the at least one other fluidic chamber.

46. A method of filling a fluidic chamber with a liquid, the method comprising:
receiving the assembly according to claim 1,
wherein the one of the inlet and the outlet of the fluidic chamber of the assembly comprises the inlet, and the other one of the inlet and the outlet of the fluidic chamber comprises the outlet, and
wherein the cross-sectional area of the volume of the fluidic chamber decreases from the transverse plane of the fluidic chamber to the outlet of the fluidic chamber; and
introducing the liquid into the inlet of the fluidic chamber, whereupon the liquid flows from the inlet of the fluidic chamber to the apex of the protrusion of the first piece via the channel formed by the protrusion,
whereupon reaching the apex of the protrusion, the liquid gradually fills the volume of the fluidic chamber such that a radius of curvature of a meniscus of the liquid increases from the apex of the protrusion to the transverse plane of the fluidic chamber, and decreases from the transverse plane of the fluidic chamber to the outlet of the fluidic chamber, but does not surpass a radius of curvature of one or more surfaces of the fluidic chamber, thereby minimizing the trapping of bubbles within the fluidic chamber during filling.

47. The method of claim 46, whereupon reaching the outlet of the fluidic chamber, the liquid exits the fluidic chamber via the outlet of the fluidic chamber.

48. A method of filling a fluidic chamber with a liquid, the method comprising:
receiving the assembly according to claim 1,
wherein the one of the inlet and the outlet of the fluidic chamber of the assembly comprises the outlet, and the other one of the inlet and the outlet of the fluidic chamber comprises the inlet, and
wherein the cross-sectional area of the volume of the fluidic chamber decreases from the transverse plane of the fluidic chamber to the inlet of the fluidic chamber; and
introducing the liquid into the inlet of the fluidic chamber, whereupon the liquid gradually fills the volume of the fluidic chamber such that a radius of curvature of a meniscus of the liquid increases from the inlet of the fluidic chamber to the transverse plane of the fluidic chamber, and decreases from the transverse plane of the fluidic chamber to the apex of the protrusion, but does not surpass a radius of curvature of one or more surfaces of the fluidic chamber, thereby minimizing the trapping of bubbles within the fluidic chamber during filling.

49. The method of claim 48, whereupon reaching the apex of the protrusion, the liquid flows into the channel formed by the protrusion and towards the outlet of the fluidic chamber, and whereupon reaching the outlet of the fluidic chamber, the liquid exits the fluidic chamber via the outlet.

50. A method of filling a fluidic chamber with a liquid, the method comprising:
receiving the assembly according to claim 11,
wherein the one of the inlet and the outlet of the fluidic chamber comprises the inlet, and the other of the one of the inlet and the outlet of the fluidic chamber comprises the outlet, and
wherein the cross-sectional area of the volume of the fluidic chamber decreases from the transverse plane to the apex of the second protrusion; and
introducing the liquid into the inlet of the fluidic chamber, whereupon the liquid flows from the inlet of the fluidic chamber to the apex of the protrusion of the first piece via the channel formed by the protrusion,
whereupon reaching the apex of the protrusion, the liquid gradually fills the volume of the fluidic chamber such that a radius of curvature of a meniscus of the liquid increases from the apex of the protrusion to the transverse plane of the fluidic chamber, and decreases from the transverse plane of the fluidic chamber to the apex of the second protrusion of the second piece, but does not surpass a radius of curvature of one or more surfaces of the fluidic chamber, thereby minimizing the trapping of bubbles within the fluidic chamber during filling.

51. A method of claim 50, whereupon reaching the apex of the second protrusion, the liquid flows into the second channel formed by the second protrusion and towards the outlet of the fluidic chamber, and
whereupon reaching the outlet of the fluidic chamber, the liquid exits the fluidic chamber via the outlet of the fluidic chamber.

52. A method of filling a fluidic chamber with a liquid, the method comprising:
receiving the assembly according to claim 11,
wherein the one of the inlet and the outlet of the fluidic chamber comprises the outlet, and the other of the one of the inlet and the outlet of the fluidic chamber comprises the inlet, and
wherein the cross-sectional area of the volume of the fluidic chamber decreases from the transverse plane to the apex of the second protrusion; and
introducing the liquid into the inlet of the fluidic chamber, whereupon the liquid flows from the inlet of the fluidic chamber to the apex of the second protrusion of the second piece via the second channel formed by the second protrusion,
whereupon reaching the apex of the second protrusion, the liquid gradually fills the volume of the fluidic chamber such that a radius of curvature of a meniscus of the liquid increases from the apex of the second protrusion to the transverse plane of the fluidic chamber, and decreases from the transverse plane of the fluidic chamber to the apex of the protrusion of the first piece, but does not surpass a radius of curvature of one or more surfaces of the fluidic chamber, thereby minimizing the trapping of bubbles within the fluidic chamber during filling.

53. The method of claim 52, whereupon reaching the apex of the protrusion, the liquid flows into the channel formed by the protrusion and towards the outlet of the fluidic chamber, and whereupon reaching the outlet of the fluidic chamber, the liquid exits the fluidic chamber via the outlet of the fluidic chamber.

54. The method of any one of claims 46-49, further comprising orienting the assembly such that the second piece is located in the direction of the force of gravity with respect to the first piece.

55. The method of claim 54, wherein the first surface of the first piece of the assembly slopes away from the second surface of the second piece at a non-zero slope towards the outlet of the fluidic chamber, and
wherein the method further comprises executing an assay within the fluidic chamber at least in part using the liquid contained within the fluidic chamber, whereupon bubbles formed during execution of the assay rise in the fluidic chamber in the direction opposite the force of gravity, and travel along the sloping first surface of the first piece of the assembly toward the outlet of the fluidic chamber, thereby removing bubbles from the fluidic chamber.

56. The method of any one of claims 46-49, further comprising orienting the assembly such that the first piece is located in the direction of the force of gravity with respect to the second piece.

57. The method of claim 56, wherein the second surface of the second piece of the assembly slopes away from the first surface of the first piece at a non-zero slope towards the apex of the protrusion of the first piece, and
wherein the method further comprises executing an assay within the fluidic chamber at least in part using the liquid contained within the fluidic chamber, whereupon bubbles formed during execution of the assay rise in the fluidic chamber in the direction opposite the force of gravity, and travel along the sloping second surface of the second piece of the assembly toward the apex of the protrusion of the first piece, thereby displacing bubbles from a center of the volume of the fluidic chamber.

58. The method of any one of claims 50-53, further comprising orienting the assembly such that the second piece is located in the direction of the force of gravity with respect to the first piece.

59. The method of claim 58, wherein the first surface of the first piece of the assembly slopes away from the second surface of the second piece at a non-zero slope towards the apex of the second protrusion of the second piece, and
wherein the method further comprises executing an assay within the fluidic chamber at least in part using the liquid contained within the fluidic chamber, whereupon bubbles formed during execution of the assay rise in the fluidic chamber in the direction opposite the force of gravity, and travel along the sloping first surface of the first piece of the assembly toward the apex of the second protrusion of the second piece, thereby displacing bubbles from a center of the volume of the fluidic chamber.

60. The method of any one of claims 50-53, further comprising orienting the assembly such that the first piece is located in the direction of the force of gravity with respect to the second piece.

61. The method of claim 60, wherein the second surface of the second piece of the assembly slopes away from the first surface of the first piece at a non-zero slope towards the apex of the protrusion of the first piece, and
wherein the method further comprises executing an assay within the fluidic chamber at least in part using the liquid contained within the fluidic chamber, whereupon bubbles formed during execution of the assay rise in the fluidic chamber in the direction opposite the force of gravity, and travel along the sloping second surface of the second piece of the assembly toward the apex of the protrusion of the first piece, thereby displacing bubbles from a center of the volume of the fluidic chamber.

62. The method of any one of claims 46-61, wherein the liquid reaches the outlet of the fluidic chamber when the volume of the fluidic chamber is substantially filled.

63. The method of any one of claims 55, 57, 59, and 61, wherein the assembly further comprises a light emitting element, and
wherein the method further comprises interrogating the liquid contained in the fluidic chamber using light that travels via an interrogation pathway that is orthogonal to the force of gravity.

64. The method of claim 63, wherein at least a portion of the second surface comprises a transparent material, and
wherein interrogating the liquid contained in the fluidic chamber using light that travels via the interrogation pathway that is orthogonal to the force of gravity comprises the light emitting element emitting light in a direction of the fluidic chamber along the interrogation pathway, through the transparent material and into the fluidic chamber.

65. The method of any one of claims 46-64, wherein the operative coupling of the first and the second pieces of the assembly forms a plurality of fluidic chambers that are in fluidic communication with one another via at least one of the inlet and the outlet of each fluidic chamber, and wherein the liquid travels between the plurality of fluidic chambers via the at least one of the inlet and the outlet of each fluidic chamber.
